(19) United States
(12) Patent Application Publication
(10) Pub. No.: US 2024/0041010 A1
(43) Pub. Date: Feb. 8, 2024

(54) GENERATION OF SURROGATE SIRES AND DAMS BY ABLATION OF ENDOGENOUS GERMLINE

(71) Applicant: University of Maryland, College Park, College Park, MD (US)

(72) Inventors: Bhanu Prakash Telugu, Laurel, MD (US); KI-EUN PARK, Reisterstown, MD (US); CHI-HUN PARK, Colombia, MO (US)

(21) Appl. No.: 18/007,156

(22) PCT Filed: Jul. 30, 2021
(86) PCT No.: PCT/US2021/043921

§ 371 Date: Jan. 27, 2023

Related U.S. Application Data
(60) Provisional application No. 62/706,091, filed on Jul. 31, 2020.
(60) Provisional application No. 62/706,410, filed on Aug. 14, 2020.

(51) Int. Cl. A01K 67/027; C12N 5/073; C12N 15/11; C12N 9/22; C12N 15/873

(52) U.S. Cl. CPC A01K 67/0271; C12N 5/0604; C12N 15/11; C12N 9/22; C12N 15/873; C12N 2310/20; C12N 2800/80; A01K 2227/105; A01K 2227/108; A01K 2227/101; A01K 2217/075; A01K 2217/054

(57) ABSTRACT

A method is provided of producing chimeric embryos and animals with exclusively donor-derived germ cells. Inactivation of a primordial germ cell specification gene results in the loss of primordial germ cells, the precursor cells for future sperm and egg, and in total loss of the endogenous germline. When complemented with embryonic cells from a desired donor, the resulting surrogate animal has all the resulting germline, and subsequent spermatogenesis, of the donor.

### CROSS-REFERENCE TO RELATED APPLICATIONS

This application claims priority to provisional applications U.S. Ser. No. 62/706,091 filed Jul. 31, 2020 and U.S. Ser. No. 62/706,410 filed Aug. 14, 2020, which are incorporated herein by reference in their entireties.

### SEQUENCE LISTING

The instant application contains a sequence listing which has been submitted in ASCII format by electronic submission and is hereby incorporated by reference in its entirety. Said ASCII copy, created on Jul. 30, 2021, is named P13292WO00_ST25.txt and is 962,907 bytes in size.

### TECHNICAL FIELD

The invention relates to chimeric animals having germ cells exclusively derived from a donor and methods for making the same.

### BACKGROUND

The generation of a genetically engineered line of animals requires targeted modifications in the germline to be successfully transmitted to the next generation. However, aggregation of genetically modified pluripotent cells from another embryo or pluripotent stem cells including embryonic stem (ES) cells or induced pluripotent stem cells (iPS) or embryonic germ cells (EGC) often results in a lack of occupation of the germline and a failure to contribute to the germline in the chimeric offspring.

Due to the difficulties in generating chimeras that show germline expression from otherwise validated ES cell lines, it is an object of the present invention to establish a method which guarantees germline transmission in the first generation by taking advantage and manipulating the genes involved in the primordial germ cells (PGC) specification pathway. Other objects will become apparent from the description of the invention which follows.

### SUMMARY

The present disclosure provides methods for producing a non-human chimeric embryo or chimeric animal with donor-derived pluripotent cells. The methods comprise providing a host embryo comprising an inactivated primordial germ cell (PGC) specification gene; and complementing the host embryo with donor cells to yield a chimeric embryo such that the germ cells of the chimeric embryo are exclusively derived from the donor cells.

In some embodiments, the methods of producing the chimeric embryo include use of a blastocyst complementation technique. In another embodiment, the methods of producing the chimeric embryo include use of an embryo-embryo aggregation technique. In some embodiments, the host embryo is complemented at the blastocyst stage. In another embodiment, the host embryo is complemented at the 4-cell stage, 6-cell stage, or 8-cell stage.

In some embodiments, the inactivated PGC specification gene is PRDM14. In another embodiment, the inactivated PGC specification gene is PRDM1, SALL4, IFITM1, DPPA3, DDX4, KITLG, DAZL, DND1, PRMT5, NANOG, AICDA, or TIAL1. The inactivation of the PGC specification gene may be accomplished by any known transgenic technique such as RNAi, or gene editing including by use of a meganuclease, a TALEN, a zinc finger nuclease, RNA-guided CRISPR-Cas, base editors, retrons, or the like. In an exemplary embodiment, the inactivation of the PGC specification gene is accomplished by injecting a zygote with a Cas protein and a guide RNA that targets the PGC specification gene.

In some embodiments, the donor cells comprise one or more pluripotent cells. In some embodiments, one or more pluripotent cells comprise embryonic stem cells, embryonic germ cells, or induced pluripotent stem cells. In another embodiment, the one or more pluripotent cells comprise a blastomere of a 4-cell stage donor embryo. In certain embodiments, the animal is a mouse, a pig, or cattle.

In some embodiments, the methods further comprise transferring the chimeric embryo into a recipient female animal; and allowing the transferred chimeric embryo to develop to term as a chimeric animal. In some embodiments, the methods further comprise breeding the chimeric animal with a second animal to produce one or more progeny animals.

Non-human chimeric embryos and chimeric animals produced by the foregoing methods are provided. Also described herein is a non-human chimeric embryo comprising host cells and donor cells. The host cells of the chimeric embryo comprise an inactivated PGC specification gene and the donor cells exclusively contribute to the germ cells of the chimeric embryo. In some embodiments, the inactivated PGC specification gene is PRDM14. In another embodiment, the inactivated PGC specification gene is PRDM1, SALL4, IFITM1, DPPA3, DDX4, KITLG, DAZL, DND1, PRMT5, NANOG, AICDA, or TIAL1. Non-human chimeric animals developed from the chimeric embryos are also provided.

While multiple embodiments are disclosed, still other embodiments of the present invention will become apparent to those skilled in the art from the following detailed description, which shows and describes illustrative embodiments of the invention. Accordingly, the figures and detailed description are to be regarded as illustrative in nature and not restrictive.

### BRIEF DESCRIPTION OF THE DRAWINGS

The following drawings form part of the specification and are included to further demonstrate certain embodiments or various aspects of the invention. In some instances, embodiments of the invention can be best understood by referring to the accompanying drawings in combination with the detailed description presented herein. The description and accompanying drawings may highlight a certain specific example, or a certain aspect of the invention. However, one skilled in the art will understand that portions of the example or aspect may be used in combination with other examples or aspects of the invention.

FIG. 1A-C is a schematic of germline specification via genome editing. Injection of wildtype embryos with CRISPR reagents to ablate PRDM14, and aggregate with GFP embryo. Chimeric surrogate sire lacking endogenous germline have exclusive contribution of gonad by donor GFP embryo. The founder animal that sires a wildtype embryo is expected to generate GFP offspring in F1 generation. This is confirmed by GFP litters in left and middle panel, compared to non-GFP age matched wildtype offspring on the right.

FIG. 2A-B shows chimeric blastocysts generated after embryo aggregation. FIG. 2A is a bright-field image. FIG. 2B is a GFP image.

FIG. 3A-C shows chimeric founder (F₀) pups born from embryo-embryo aggregations. FIG. 3A shows a chimeric pup from replicate 1. FIG. 3B shows a chimeric pup from replicate 2. FIG. 3C shows wild-type, age-matched control pups.

FIG. 4A-B shows chimeric founder (F₀) pups born from blastocyst complementation with R1 cells. FIG. 4A shows chimeric pups from replicate 1. FIG. 4B shows a chimeric pup from replicate 2.

FIG. 5 shows two representative F₁ litters generated from each of the R1 chimera founder males. Lack of GFP expression indicates germline occupied solely by ESC background.

FIG. 6A-C shows founder (F₀) pups born from blastocyst complementation with Cwc15^{-/-} cells. FIG. 6A shows pups from replicate 1. FIG. 6B shows a pup from replicate 2. FIG. 6C shows F₁ pups from mating of F₀ to wild-type partners. Stars indicate founder chimeras that have low levels of chimerism.

FIG. 7A-B shows gene expression of POU5F1 and NANOG at varying stages of embryo development. Least-square means of the natural log of gene copy number±SE are presented. Different letters indicate that values are significantly different (p<0.05).

FIG. 8A-B shows gene expression of SOX2 and ESRRB at varying stages of embryo development. Least-square means of the natural log of gene copy number±SE are presented. Different letters indicate that values are significantly different (p<0.05).

FIG. 9A-B shows gene expression of PRDM14 and PRDM1 at varying stages of embryo development. Least-square means of the natural log of gene copy number±SE are presented. Different letters indicate that values are significantly different (p<0.05).

FIG. 10 shows gene expression of TFAP2C at varying stages of embryo development. Least-square means of the natural log of gene copy number±SE are presented. Values are not significantly different (p>0.05).

FIG. 11A-B shows gene expression of DAZL and VASA at varying stages of embryo development. Least-square means of the natural log of gene copy number±SE are presented. Different letters indicate that values are significantly different (p<0.05).

FIG. 12 shows gene expression of STRA8 at varying stages of embryo development. Least-square means of the natural log of gene copy number±SE are presented. Values are not significantly different (p>0.05).

FIG. 13A-B shows gene expression of CARM1 and TET1 at varying stages of embryo development. Least-square means of the natural log of gene copy number±SE are presented. Different letters indicate that values are significantly different (p<0.05).

FIG. 14 shows gene expression of TET2 at varying stages of embryo development. Least-square means of the natural log of gene copy number±SE are presented. Different letters indicate that values are significantly different (p<0.05).

FIG. 15 shows a schematic outline of the surrogate sires and dams technology. Recipient embryos from a generic herd are knocked out for PGC specification gene, PRDM14, and the resultant embryo is aggregated with “donor” embryonic cells from elite animals or genome edited founder, whose genetics needs to be preserved and/or amplified for amplifying genetic gains. When the reconstituted embryos are transferred into synchronized recipient animals the resultant offspring are chimeric for somatic lineage, but the germline is exclusively from the donor animals. Because the supporting nurse cells are largely intact and unperturbed by the genetic modification, robust donor-derived spermatogenesis and oogenesis is expected in the resultant animals.

FIG. 16A-D shows generation and characterization of PRDM14 null pig fetuses. FIG. 16A is a schematic outlining the targeting strategy. In pig, the long isoform of PRDM14 is coded by 7 exons. Exon 4 represents the first common coding exon in all isoforms, and hence was targeted. A targeting oligo containing 100 bp of homology flanking the cut site and containing the “TAG” stop codon in the middle was designed such that successful gene targeting will result in the insertion of the stop codon and a “T” in the PAM motif, resulting in the knockout of the gene, and disruption of the PAM motif, such that future cuts at the targeted site will be thwarted. FIG. 16B shows the results from “targeting amplicon sequencing” with primers flanking the CRISPR cut site. The amplicons were sequenced using in-house Illumina iSeq, and the reads aligned to the putative modified allele using CRISPRESSO 2.0 software (SEQ ID NOs: 107-110). Results from representative clonal lines show greater than 96% of the reads aligning to the modified knockout allele. FIG. 16C shows RT-PCR confirmed the loss of PRDM14 and another germ cell specific transcript, DAZL in fetuses cloned from the targeted PRDM14 knockout colonies. FIG. 16D shows immunohistochemistry with antibody for PRDM14 confirmed the loss of germ cells in the fetuses.

### DETAILED DESCRIPTION OF THE PREFERRED EMBODIMENTS

Inactivation of a primordial germ cell (PGC) specification gene such as PRDM14 or PRDM1 results in the loss of PGC. When complemented with pluripotent cells from a desired donor, the resulting surrogate animal has all the resulting germline (and subsequent spermatogenesis) from the donor derived cells. One advantage of this approach is that the supporting cells originating from the host embryo are largely intact, and when the donor PGCs reach the gonad, the resulting offspring will have established robust spermatogenesis or oogenesis. The resulting surrogate sires or dams will ensure that hard earned genetic gain is preserved and amplified for robust dissemination of genetics for subsequent generations.

This approach is unique from somatic cell nuclear transfer (current paradigm) approaches for preserving the rare genetic lottery in that the majority of the embryonic cells and consequently the resulting offspring is of the host (PGC specification gene knockout) embryonic origin, with the exception of germline, which is contributed exclusively by the donor cells. Many of the drawbacks associated with somatic cell nuclear transfer such as low pregnancy/maintenance rates and altered epigenetics can be overcome, whilst ensuring robust gametogenesis for propagation/dissemination of donor genetics.

So that the present invention may be more readily understood, certain terms are first defined. Unless defined otherwise, all technical and scientific terms used herein have the same meaning as commonly understood by one of ordinary skill in the art to which embodiments of the invention pertain. Many methods and materials similar, modified, or equivalent to those described herein can be used in the practice of the embodiments of the present invention without undue experimentation, the preferred materials and methods are described herein. In describing and claiming the embodiments of the present invention, the following terminology will be used in accordance with the definitions set out below.

The singular terms “a”, “an”, and “the” include plural referents unless context clearly indicates otherwise. Similarly, the word “or” is intended to include “and” unless the context clearly indicate otherwise. The word “or” means any one member of a particular list and also includes any combination of members of that list.

Numeric ranges recited within the specification, including ranges of “greater than,” “at least”, or “less than” a numeric value, are inclusive of the numbers defining the range and include each integer within the defined range. For example, when a range of “1 to 5” is recited, the recited range should be construed as including ranges “1 to 4”, “1 to 3”, “1-2”, “1-2 & 4-5”, “1-3 & 5”, and the like.

As used herein “blastocyst” means an early developmental stage of embryo comprising of inner cell mass (from which embryo proper arises) and a fluid filled cavity typically surrounded by a single layer of trophoblast cells. “Developmental Biology”, sixth edition, ed. by Scott F. Gilbert, Sinauer Associates, Inc., Publishers, Sunderland, Mass. (2000).

The term “blastocyst complementation” as used herein refers to a technique for creating a chimeric animal in which injection of multipotent or pluripotent cells, such as ES cells and iPS cells, into an inner space of a blastocyst stage fertilized egg forms a chimeric animal when implanted into a female for gestation (e.g., pseudo-pregnant or pregnant female).

The term “chimeric blastocyst” as used herein refers to a blastocyst that comprises cellular material from a pluripotent cell derived from a different source than that of the blastocyst.

As used herein, the term “cow” or “cattle” is used generally to refer to an animal of bovine origin of any age or gender. Interchangeable terms include “bovine”, “calf”, “steer”, “bull”, “heifer” and the like. It also includes an individual animal in all stages of development, including embryonic and fetal stages.

The term “early stage embryo” means any embryo at embryonic stages between fertilized ovum and blastocyst. Typically, eight cell stage and morula stage embryos are referred to as early stage embryos.

“Embryonic germ cells” or “EG cells” means primordial germ cell derived cells which have the potential to differentiate into all the cell types of body and are as amenable to genetic modification as embryonic stem cells, to the extent that sometimes the distinction between EG cells and ES cells is ignored. “Developmental Biology”, sixth edition, ed. by Scott F. Gilbert, Sinauer Associates, Inc., Publishers, Sunderland, Mass. (2000).

“Embryonic stem cells” or “ES cells” means cultured cells derived from inner cell mass of early stage embryo, which are amenable to genetic modification and which retain their totipotency and can contribute to all organs of resulting chimeric animal if injected into host embryo. “Developmental Biology”, sixth edition, ed. by Scott F. Gilbert, Sinauer Associates, Inc., Publishers, Sunderland, Mass. (2000).

As used herein, “fertilization” means the union of male and female gametes during reproduction resulting into formation of zygote, the earliest developmental stage of an embryo.

“Germ cell development” means the process by which certain cells in the early stage developing embryo differentiate into primordial germ cells.

“Germ cell migration” means the process by which primordial germ cells, after originating in the extraembryonic mesoderm travel back in the embryo through allantois (precursor of umbilical cord) and continue to migrate through adjacent yolk sac, hindgut, and dorsal mesentery to finally reach the genital ridge (developing gonad). “Developmental Biology”, sixth edition, ed. by Scott F. Gilbert, Sinauer Associates, Inc., Publishers, Sunderland, Mass. (2000).

“Germ line cell” means any cell, at any stage of differentiation towards mature gametes, including mature gametes.

“Primordial germ cells” means those cells arising early in the embryonic development that give rise to the spermatogenic lineage via a gonocyte intermediate or female germline via an oogonia intermediate.

As used herein, the terms nucleic acid or polynucleotide refer to deoxyribonucleotides or ribonucleotides and polymers thereof in either single- or double-stranded form unless indicated otherwise. As such, the terms include RNA and DNA, which can be a gene or a portion thereof, a cDNA, a synthetic polydeoxyribonucleic acid sequence, or the like, and can be single-stranded or double-stranded, as well as a DNA/RNA hybrid. Unless otherwise indicated, a particular nucleic acid sequence also implicitly encompasses conservatively modified variants thereof (e.g. degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al. (1991) Nucleic Acid Res. 19:5081; Ohtsuka et al. (1985) J. Biol. Chem. 260:2605-2608; Rossolini et al. (1994) Mol. Cell. Probes 8:91-98). The term nucleic acid is used interchangeably with gene, cDNA, and mRNA encoded by a gene.

A “polypeptide” refers generally to peptides and proteins. In certain embodiments the polypeptide may be at least two, three, four, five, six, seven, eight, nine or ten or more amino acids or more or any amount in-between. A peptide is generally considered to be more than fifty amino acids. The terms “fragment,” “derivative” and “homologue” when referring to the polypeptides according to the present invention, means a polypeptide which retains essentially the same biological function or activity as said polypeptide. Such fragments, derivatives and homologues can be chosen based on the ability to retain one or more of the biological activities of the polypeptide. The polypeptides may be recombinant polypeptides, natural polypeptides or synthetic polypeptides.

“Codon optimization” can be used to optimize sequences for expression in an animal and is defined as modifying a nucleic acid sequence for enhanced expression in the cells of the animal of interest, e.g. swine, by replacing at least one, more than one, or a significant number, of codons of the native sequence with codons that are more frequently or most frequently used in the genes of that animal. Various species exhibit particular bias for certain codons of a particular amino acid. Cas9 can be one of the sequences codon optimized for improved expression.

In one aspect, polynucleotides comprising nucleic acid fragments of codon-optimized coding regions which may produce RNA, encode polypeptides, or fragments, variants, or derivatives thereof, with the codon usage adapted for optimized expression in the cells of a given animal. These polynucleotides are prepared by incorporating codons preferred for use in the genes of the host of interest into the DNA sequence.

A “heterologous” nucleic acid molecule is any which is not naturally found next to the adjacent nucleic acid molecule. A heterologous polynucleotide or a heterologous nucleic acid or an exogenous DNA segment refers to a polynucleotide, nucleic acid or DNA segment that originates from a source foreign to the particular host cell, or, if from the same source, is modified from its original form in composition and/or genomic locus by human intervention. A heterologous gene in a host cell includes a gene that is endogenous to the particular host cell, but has been modified or introduced into the host. Thus, the terms refer to a nucleic acid molecule which is foreign or heterologous to the cell, or homologous to the cell but in a position within the host cell nucleic acid in which the element is not ordinarily found.

A nucleic acid may then be introduced into an animal host cell through the use of a vector, plasmid or construct and the like. A “vector” is any means for the transfer of a nucleic acid into a host cell. Vectors can be single stranded, double stranded or partially double stranded, may have free ends or no free ends, may be DNA, RNA or both. A variety of polynucleotides are known to be useful as vectors. A plasmid is a circular double stranded DNA loop. Referring to one or more expression vectors is meant to refer to one or more vectors comprising necessary regulatory elements for proper expression of the operably linked nucleic acid molecules. A vector may be a replicon to which another DNA segment may be attached so as to bring about the replication of the attached segment. A replicon is any genetic element (e.g., plasmid, phage, cosmid, chromosome, virus) that functions as an autonomous unit of DNA or RNA replication in vivo, i.e., capable of replication under its own control. The term “vector” includes both viral and nonviral means for introducing the nucleic acid into a cell in vitro, ex vivo or in vivo. Viral vectors include but are not limited to adeno-associated viruses, lentiviruses, alphavirus, retrovirus, pox, baculovirus, vaccinia, herpes simplex, Epstein-Barr, rabies virus, and vesicular stomatitis virus. Non-viral vectors include, but are not limited to plasmids, liposomes, electrically charged lipids (cytofectins), DNA- or RNA protein complexes, and biopolymers. In addition to a nucleic acid, a vector may also contain one or more regulatory regions, and/or selectable markers useful in selecting, measuring, and monitoring nucleic acid transfer results (transfer to which tissues, duration of expression, etc.). Transformed cells can be selected, for example, by resistance to antibiotics conferred by genes contained on the plasmids, such as the amp, kan, gpt, neo and hyg genes. The techniques employed to insert such a sequence into the viral vector and make ether alterations in the viral DNA, e.g., to insert linker sequences and the like, are known to one of skill in the art. (See, e.g., Sambrook et al., 2001. Molecular Cloning: A Laboratory Manual, 3^{rd} Edition. Cold Spring Harbor Laboratory Press, Plainview, NY). A “cassette” refers to a segment of DNA that can be inserted into a vector at specific restriction sites. The segment of DNA encodes a polypeptide of interest or produces RNA, and the cassette and restriction sites are designed to ensure insertion of the cassette in the proper reading frame for transcription and translation.

The nucleic acid molecule may be operably linked to a suitable promoter at the 5′ end and a termination signal and poly(A) signal at the 3′ end. As used herein, the term “operably linked” means that the nucleic acid molecule containing an expression control sequence, e.g., transcription promoter and termination sequences, are situated in a vector or cell such that expression of the polypeptide or RNA produced by the nucleic acid molecule is regulated by the expression control sequence. Methods for cloning and operably linking such sequences are well known in the art. Promoters may direct constitutive expression or tissue preferred expression. Tissue-preferred (sometimes called tissue-specific) promoters can be used to target enhanced transcription and/or expression within a particular cell or tissue. Such promoters express at a higher level in the particular cell region or tissue than in other parts of the cell or tissue and may express primarily in the cell region or tissue. Examples include promoters that secrete to the cell wall, retain expression in the endoplasmic reticulum, or target vacuoles or other cell organelles. Other may direct expression primarily to muscle, neuron, bone, skin, blood or specific organs or cell types. Such promoters may also direct expression in a temporal manner, expressing at a particular stage of development or cycle of the cell. The promoter(s) utilized in one example may be polymerase (pol) I, pol II or pol III promoters. Examples of pol I promoters include the chicken RNA pol I promoter. Examples of pol II promoters include but are not limited to the cytomegalovirus immediate-early (CMV) promoter, the Rous sarcoma virus long terminal repeat (RSV-LTR) promoter, and the simian virus 40 (SV40) immediate-early promoter. Examples of pol III promoters includes U6 and H1 promoters. Inducible promoters may be used such as the metallothionein promoter. Other examples of promoters include, T7 phage promoter, T3 phage promoter, beta-galactosidase promoter, and the Sp6 phage promoter. An example of a DNA having a termination and poly(A) signal is the SV40 late poly(A) region. The use of these commercially available expression vectors and systems are well known in the art. The vector may contain multiple copies of a nucleic acid molecule of interest or a combination of nucleic acid molecules; also multiple vectors may be introduced simultaneously or sequentially into the cell.

Other components may be included in the vector or in vectors also introduced into the cells, such as polyadenylation sequences, enhancers, signal peptides, inducible elements, introns, translation control sequences or the like. As noted above, selectable markers allowing survival of cells with the vector or other identification of cells having the vector may be used.

A nucleic acid molecule is introduced into a cell when it is inserted in the cell. A cell has been “transfected” by exogenous or heterologous DNA or RNA when such DNA or RNA has been introduced inside the cell. When referring to integration of a nucleic acid molecule into a cell is meant that the molecule has recombined and become part of the genome.

The presence of the nucleic acid molecule may be determined by any convenient technique, such as identifying the presence of a marker gene; detecting the presence of the inserted sequence via PCR or the like; detecting expression product from animal cells, tissue or fluids; Northern or Western blot analysis; or any other readily available method.

### Primordial Germ Cell (PGC) Specification

PGCs are specialized cells that are the precursors of gametes. PGC are responsible for passing on genetic information from parent to offspring through generations in order to ensure survival of a species. These cells are specified very early in development from a subset of mesodermal cells which originate at the primitive streak. Due to their short generation interval and fast developmental timeline, many studies on PGC specification and development have been focused on the mouse model system, with only a few published studies on PGC specification and commitment events in other mammals. Any gene involved PGC may be used according to the invention.

In the mouse, progenitors of PGCs arise from the posterior region of the post-implantation epiblast. At the onset of gastrulation, a cluster of approximately 40 PGCs arise from bone morphogenetic protein (BMP) signals secreted by neighboring cells. Precursors of PGCs are induced by BMP signaling (BMP2, BMP4, and BMP8b) from cells in the extraembryonic ectoderm (ExE). These BMPs act through SMAD1 and SMAD5 signaling to induce expression of PR domain containing 1 (PRDM1) and PRDM14 in a dose-dependent manner, with the highest levels of BMP occurring in the posterior proximal epiblast. Restriction of PGCs to the posterior epiblast location occurs due to BMP inhibitory signals such as left-right determination factor 1 (LEFTY1), cerberus 1 (CER1), and dickkopf homolog 1 (DKK1), which prevent posteriorization of the anterior epiblast. Activation of PRDM1 in precursor cells as early as E6.25, initiates a cascade of events including the induction of PRDM14 and transcription factor AP2-gamma (TFAP2C) in PGC precursors that lead to PGC specification. Because the PGC are derived from a mesodermal population, PGC precursors also initially express mesodermal transcripts such as homeobox (Hox) genes and brachyury (T). However, after the induction of PRDM1, PRDM14, and TFAP2C, the HOX genes are repressed and pluripotency genes POU5F1, SOX2, and NANOG are expressed.

After PGCs are specified, PRDM1, PRDM14, and TFAP2C then coordinately form a network which is able to repress the somatic program, induce genome-wide epigenetic reprogramming, and initiate the reacquisition of pluripotency. Each of these three genes has a unique role in accomplishing these three goals. Prdm1 is responsible for repression of the somatic program, although its exact method of action is not clearly understood. Prdm14 is absolutely essential in PGC specification and is involved in epigenetic reprogramming as well as initiating and maintaining pluripotency, even in ESC in culture. Tfap2c is believed to function downstream of Prdm1 and is known to be important for migration of PGCs to the gonad because knockouts show reduced cell number and PGCs fail to migrate. Tfap2c mutants are able to specify the initial PGC population but further germ cell differentiation is impaired, and somatic differentiation is initiated.

In the pig, progenitors of PGC arise in the caudal third of the embryo scattered around the primitive streak at day 12 of embryonic. By day 13, the progenitors are still in the area of the primitive streak though some have appeared in the extra-embryonic yolk sac wall, forming a cluster of PGCs. These progenitor cells are characterized by continued expression of POU5F1 after the epiblast has ceased its expression of POU5F1. They also express SOX17, and most cells within the cluster also express PRDM1. In cells that express both SOX17 and PRDM1, NANOG expression is also retained from the early epiblast.

Between E12.5 and E13.5, PGCs exhibit co-expression of a variety of pluripotency and PGC factors: SOX17, PRDM1, NANOG, TFAP2C, and OCT4, as determined by immunohistochemical staining. At this stage of development, porcine PGCs do not express the mesodermal factor T. Surprisingly, PRDM14 expression is weak during this specification period, and appears cytoplasmic at E14. As in mice, the initial PGC cluster (E12) contains few cells (˜60) which soon increase to more than 300 cells by E 15.5. During E14-15, the yolk sac folds under the posterior portion of the embryo to form the ventral wall of the hind gut. The PGCs then become restricted to this area at E15 and can be found in the entire length of the hind gut. After the sharp increase in PGC number, they enter quiescence prior to migration, similar to the mouse system.

In certain embodiments, the inactivated PGC specification gene is PRDM14, PRDM1, SALL4, IFITM1, DPPA3, DDX4, KITLG, DAZL, DND1, PRMT5, NANOG, AID/AICDA, TIAR/TIAL1, or a combination thereof. Inactivating PGC specification genes encoding proteins with the amino acid sequences listed in Table 1 (SEQ ID NOs: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, and 78), or sequences with at least 70%, at least 80%, at least 90%, at least 95%, at least 96%, at least 97%, at least 98%, or at least 99% sequence identity to the protein sequences listed in Table 1, may result in the loss of PGC specification and/or the lack of ability for functional spermatogenesis or oogenesis.

TABLE 1

| Gene | Species | Genomic sequence | Protein sequence |
|---|---|---|---|
| PRDM14 | Mus musculus | SEQ ID NO: 1 | SEQ ID NO: 2 |
| | Sus scrofa | SEQ ID NO: 3 | SEQ ID NO: 4 |
| | Bos taurus | SEQ ID NO: 5 | SEQ ID NO: 6 |
| PRDM1 | Mus musculus | SEQ ID NO: 7 | SEQ ID NO: 8 |
| | Sus scrofa | SEQ ID NO: 9 | SEQ ID NO: 10 |
| | Bos taurus | SEQ ID NO: 11 | SEQ ID NO: 12 |
| SALL4 | Mus musculus | SEQ ID NO: 13 | SEQ ID NO: 14 |
| | Sus scrofa | SEQ ID NO: 15 | SEQ ID NO: 16 |
| | Bos taurus | SEQ ID NO: 17 | SEQ ID NO: 18 |
| IFITM1 | Mus musculus | SEQ ID NO: 19 | SEQ ID NO: 20 |
| | Sus scrofa | SEQ ID NO: 21 | SEQ ID NO: 22 |
| | Bos taurus | SEQ ID NO: 23 | SEQ ID NO: 24 |
| DPPA3 | Mus musculus | SEQ ID NO: 25 | SEQ ID NO: 26 |
| | Sus scrofa | SEQ ID NO: 27 | SEQ ID NO: 28 |
| | Bos taurus | SEQ ID NO: 29 | SEQ ID NO: 30 |
| DDX4 | Mus musculus | SEQ ID NO: 31 | SEQ ID NO: 32 |
| | Sus scrofa | SEQ ID NO: 33 | SEQ ID NO: 34 |
| | Bos taurus | SEQ ID NO: 35 | SEQ ID NO: 36 |
| KITLG | Mus musculus | SEQ ID NO: 37 | SEQ ID NO: 38 |
| | Sus scrofa | SEQ ID NO: 39 | SEQ ID NO: 40 |
| | Bos taurus | SEQ ID NO: 41 | SEQ ID NO: 42 |
| DAZL | Mus musculus | SEQ ID NO: 43 | SEQ ID NO: 44 |
| | Sus scrofa | SEQ ID NO: 45 | SEQ ID NO: 46 |
| | Bos taurus | SEQ ID NO: 47 | SEQ ID NO: 48 |
| DND1 | Mus musculus | SEQ ID NO: 49 | SEQ ID NO: 50 |
| | Sus scrofa | SEQ ID NO: 51 | SEQ ID NO: 52 |
| | Bos taurus | SEQ ID NO: 53 | SEQ ID NO: 54 |
| PRMT5 | Mus musculus | SEQ ID NO: 55 | SEQ ID NO: 56 |
| | Sus scrofa | SEQ ID NO: 57 | SEQ ID NO: 58 |
| | Bos taurus | SEQ ID NO: 58 | SEQ ID NO: 60 |
| NANOG | Mus musculus | SEQ ID NO: 61 | SEQ ID NO: 62 |
| | Sus scrofa | SEQ ID NO: 63 | SEQ ID NO: 64 |
| | Bos taurus | SEQ ID NO: 65 | SEQ ID NO: 66 |
| AID/AICDA | Mus musculus | SEQ ID NO: 67 | SEQ ID NO: 68 |
| | Sus scrofa | SEQ ID NO: 69 | SEQ ID NO: 70 |
| | Bos taurus | SEQ ID NO: 71 | SEQ ID NO: 72 |
| TIAR/TIAL1 | Mus musculus | SEQ ID NO: 73 | SEQ ID NO: 74 |
| | Sus scrofa | SEQ ID NO: 75 | SEQ ID NO: 76 |
| | Bos taurus | SEQ ID NO: 77 | SEQ ID NO: 78 |

### PGC Specification Gene Inactivation

Any method which provides for inactivation of the PGC specification gene may be utilized. The term “inactivation” includes any method that prevents the functional expression of one or more PGC specification genes such that the gene or gene product is unable to exert its known function. Means of gene inactivation include deletions, disruptions of the protein-coding sequence, insertions, additions, mutations, gene silencing (e.g. RNAi) and the like.

The following is provided by way of example rather than limitation. A guide nucleic acid molecule is one that directs the nuclease to the specific cut site in the genome, whether via use of a binding domain, recognition domains, guide RNAs or other mechanisms. The guide nucleic acid molecule is introduced into the cell under conditions appropriate for operation of the guide nucleic acid molecule in directing cleavage to the target locus. A person of skill in the art will have available a number of methods that may be used, the most common utilizing a nuclease to cleave the target region of the gene, along with sequences which will recognize sequences at the target locus and direct cleavage to the locus. Any nuclease that can cleave the phosphodiester bond of a polynucleotide chain may be used in the methods described here. By way of example without limitation, available systems include those utilizing site specific nucleases (SSN) such as ZFNs (Zinc finger nuclease), (Whyte, J. J. et al. Gene targeting with zinc finger nucleases to produce cloned eGFP knockout pigs. Mol Reprod Dev 78, 2 (2011); Whyte, et al. Cell Biology Symposium: Zinc finger nucleases to create custom-designed modifications in the swine (Sus scrofa) genome. J Anim Sci 90, 1111-1117 (2012)); TALENs (Transcription activator-like effector nucleases) (see, Carlson, D. F. et al. Efficient TALEN-mediated gene knockout in livestock. Proc Natl Acad Sci USA 109, 17382-17387 (2012); Tan, W. et al. Efficient nonmeiotic allele introgression in livestock using custom endonucleases. Proc Natl Acad Sci USA 110, 16526-16531 (2013); Lillico, S. G. et al. Live pigs produced from genome edited zygotes. Scientific reports 3, 2847 (2013)), and the CRISPR (Clustered regularly interspaced short palindromic repeats)-associated (Cas) nuclease system (Hai, T., Teng, F., Guo, R., Li, W. & Zhou, Q. One-step generation of knockout pigs by zygote injection of CRISPR/Cas system. Cell Res 24, 372-375 (2014)) that have permitted editing of animal genomes such as pig genomes with relative ease. The use of recombinases such as FLP/FRT as described in U.S. Pat. No. 6,720,475, or CRE/LOX as described in U.S. Pat. No. 5,658,772, can be utilized to integrate a polynucleotide sequence into a specific chromosomal site. Meganucleases have been used for targeting donor polynucleotides into a specific chromosomal location as described in Puchta et al., PNAS USA 93 (1996) pp. 5055-5060. ZFNs work with proteins or domains of proteins binding to a binding domain having a stabilized structure as a result of use a zinc ion. TALENs utilize domains with repeats of amino acids which can specifically recognize a base pair in a DNA sequence. For a discussion of both systems see Voytas et al. U.S. Pat. No. 8,697,853, incorporated herein by reference in its entirety. These systems utilize enzymes prepared for each target sequence.

The CRISPR/Cas nuclease system has evolved in archaea and bacteria as an RNA based adaptive immunity system to detect and cleave invading viruses and plasmids. (Horvath, P. & Barrangou, R. CRISPR/Cas, the immune system of bacteria and archaea. Science 327, 167-170 (2010); Wiedenheft, et al. RNA-guided genetic silencing systems in bacteria and archaea. Nature 482, 331-338 (2012)). Unlike ZFNs and TALENs, which require assembly of DNA binding domain (DBD) to direct the nuclease to the target site, the CRISPR/Cas system utilizes RNA as a guide. The CRISPR locus is a distinct class of interspersed short sequence repeats (SSRs) recognized in bacterial genes. The repeats are short elements occurring in clusters that are regularly spaced by unique intervening sequences with a substantially constant length. They were observed as an immunity system in which nucleic acid molecules homologous to virus or plasmid sequences are integrated into the CRISPR loci. The foreign DNA or RNA is targeted and cleaved. The system has been adapted for targeted insertion of a nucleic acid molecule at a defined locus. In general, a CRISPR system is characterized by elements that promote the formation of a CRISPR complex at the site of a target sequence to which a guide sequence is designed to have complementarity, where hybridization between a target sequence and a guide sequence promotes the formation of a CRISPR complex. Full complementarity is not necessarily required, provided there is sufficient complementarity to cause hybridization and promote formation of a CRISPR complex. In the CRISPR system one enzyme, a CRISPR enzyme is used for targeting using short RNA molecules.

Two basic components are used with the system, a guide RNA and an endonuclease. The guide RNA is endogenous sequence specifying the target site and tracrRNA, needed to bind to the enzyme. The guide sequence provides target specificity and the tracrRNA provides scaffolding properties. These guide sequences are typically about 15 up to 20 to 25 base pairs (bp) that hybridize with the target site and direct binding of a CRISPR complex to a target sequence. A sequence encoding a CRISPR-associated enzyme may be provided on the same or different vectors. Non-limiting examples of Cas proteins include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof. In one embodiment the enzyme is a type II CRISPR system enzyme and is Cas9 or variants or modifications thereof. These enzymes are known; for example, the amino acid sequence of S. pyogenes Cas9 protein may be found in the SwissProt database under accession number Q99ZW2. The enzyme or Cas9 protein can be used as a nickase or nuclease and cleave one or two strands of DNA. Cas9 has two functional domains, RuvC and HNH and when both are used both strands are cleaved. Cas9 nuclease forms a ribonuclease complex with target CRISPR RNAs (crRNAs) and transactivating RNAs (tracrRNA), and uses the chimeric RNA to target the genomic sequence and induce DSB. The CRISPR/Cas nuclease and other SSN can introduce a targeted double strand break (DSB) in the genomic DNA, which in the presence of a single stranded (SS) DNA oligonucleotide or a double stranded (DS) targeting vector, result in homologous recombination (HR) based alteration of selected nucleotides or KI of transgenes respectively, into the target loci. In another embodiment a SS oligonucleotide having the nucleic acid molecule of interest may be used with Cas9 mRNA and sgRNA to target modification of a particular target gene region. In further embodiments the target gene is complementary to the gRNA sequence and will have a protospacer adjacent motif or PAM sequence. This aids in binding by Cas9. For a discussion of details of the CRISPR/Cas system see Cong et al., U.S. Pat. Nos. 8,932,814; 8,871,445 and 8,906,616, incorporated by reference herein in their entirety.

Breaks in the genome can be repaired by the non-homologous end joining DNA repair pathway (NHEJ) or by homology directed repair pathway (HDR). NHEJ can disrupt the gene, by causing frame shifts or premature stop codons to occur. HDR is an embodiment that provides for insertion of a nucleic acid molecule that avoids such issues. With a double strand break a DNA repair template is provided in which sequences are provided that have homology to and hybridize with genome sequences flanking the cleavage site (homology arm). In one embodiment the DNA template or flanking sequences are transfected into the cell with the CRISPR/Cas vector.

Even though HDR-based gene targeting events are extremely rare, the efficiencies can be improved by several orders of magnitude (>1000-fold) by introducing a DSB at the target locus (Moehle, E. A. et al. Targeted gene addition into a specified location in the human genome using designed zinc finger nucleases. Proc Natl Acad Sci USA 104, 3055-3060 (2007)). Following DSB, either a SS oligo, or a DS vector with homology to the ends flanking the DSB, can produce animals with targeted genomic alterations or transgene integrations (Cui, L et al. The permissive effect of zinc deficiency on uroguanylin and inducible nitric oxide synthase gene upregulation in rat intestine induced by interleukin 1alpha is rapidly reversed by zinc repletion. The Journal of Nutrition 133, 51-56 (2003); Meyer, M et al. Gene targeting by homologous recombination in mouse zygotes mediated by zinc-finger nucleases. Proc Natl Acad Sci USA 107, 15022-15026 (2010)).

A still further example provides for introduction into the animal cell of interfering nucleic acid molecules. For example, double-stranded RNA molecules (dsRNA) may be employed. In this process, in summary, RNA which is double stranded, in part, or completely, is produced based upon the sequence of the target nucleic acid molecule. Specifics of the means of producing the dsRNA may vary as one skilled in the art appreciates, and include, by way of example without intending to be limiting, the approach of Graham et al., U.S. Pat. No. 6,573,099 where two copies of a sequence corresponding to a target sequence is used, or that of Fire et al., U.S. Pat. No. 6,326,193 (both incorporated herein by reference), where the first strand is an RNA sequence corresponding to the target nucleic acid, and the second is one which is complementary to the target sequence, each of which are incorporated herein by reference in their entirety. These strands hybridize with each other to form the inhibiting dsRNA. The strand which corresponds to the target nucleic acid molecule can correspond to all or a portion thereof, as long as a dsRNA is formed. Where a strand is used which is the complement (antisense) of the target nucleic acid is used, it can be complementary to all or a portion of the target nucleic acid molecule, so long as the dsRNA formed interferes with the target nucleic acid molecule. The dsRNA triggers a response in which the RNAse III Dicer enzyme process dsRNA into small interfering RNAs (siRNA) of approximately 21-23 nucleotides, which are formed into a RNA-induced silencing complex RISC which destroys homologous mRNAs. (See, Hammond, S. M., et al., Nature (2000) 404:293-296). Generally, sequences of up to 10 nucleotides 20 nucleotides, 30 nucleotides, 40 nucleotides, 50 nucleotides, 100 nucleotides, 200 nucleotides, 300, 500, 550, 500, 550, or greater and any amount in-between may be used.

In referring to injection in the context of inserting a nucleic acid or a protein into a cell, it is meant any convenient method of inserting a device into the cell and passage of the nucleic acid molecules or proteins into the cell. By way of example without limitation, this can be accomplished with an injection pipette which may include a syringe holding the nucleic acid molecules or proteins.

### Complementation

The ablation of endogenous germline may be complemented by a suitable method including blastocyst complementation or embryo-embryo aggregation. In blastocyst complementation, donor stem cells are injected into the host embryo at blastocyst stage, as is the usual technique for generating chimeras. In embryo-embryo aggregation, one blastomere of a 4-cell stage donor embryo is injected into a 4-cell host embryo of a different strain background.

Recently, blastocyst complementation has become a popular technique to direct cells toward a specific lineage. This technique is coming of use for biomedical applications, as researchers are seeking a way to grow human organs in other species for potential use as transplants. Much evidence has been presented in the mouse model that this technique works, but interspecies chimeras are still in development.

Blastocyst complementation is also being used to generate interspecies chimeras, with the hope for future human applications. The biomedical field is rapidly developing techniques to attempt generating human organs inside large animals, most notably the pig. Introductory studies using the mouse and rat to determine the feasibility of interspecies chimeras have determined that this technique is possible. Rat-mouse chimeras have been reported, with a rat pancreas generated in a Pdx1-null mouse, and the reverse experiment as well. The first report of human-pig chimeras shows that it is possible to have human iPSC incorporated into a porcine fetus up until E28, albeit at low frequency and low levels of chimerism.

The methods may be used in any animal. Suitable animals include, but are not limited to, a human, a livestock animal, a companion animal, a lab animal, and a zoological animal. In one embodiment, the subject may be a rodent, e.g. a mouse, a rat, a guinea pig, etc. In another embodiment, the subject may be a livestock animal. Non-limiting examples of suitable livestock animals may include pigs, cows, horses, goats, sheep, llamas and alpacas. In certain embodiments, the animal is a is an ungulate or a ruminant animal. In yet another embodiment, the subject may be a companion animal. Non-limiting examples of companion animals may include pets such as dogs, cats, rabbits, and birds. In some embodiments, the animal is a laboratory animal. Non-limiting examples of a laboratory animal may include rodents, canines, felines, and non-human primates. In certain embodiments, the animal is a rodent. Non-limiting examples of rodents may include mice, rats, guinea pigs, etc. In certain embodiments, the animal is a bovine animal. In certain embodiments, the animal is cattle. In certain embodiments, the animal is a pig.

Pig is an economically important agricultural animal. Additionally, pigs are coveted for their biomedical applications. Similar to humans and mouse, the pigs are mono-gastric, and as such are playing a dominant role in investigations of nutrient uptake, trafficking and metabolism. (Patterson, et al. The pig as an experimental model for elucidating the mechanisms governing dietary influence on mineral absorption. Experimental biology and medicine 233, 651-664 (2008)). Advances in the field of animal genome editing have included sequencing of pig genome. (Groenen, M. A. et al. Analyses of pig genomes provide insight into porcine demography and evolution. Nature 491, 393-398 (2012)). Taken together, depending on the biological question that needs to be addressed, a suitable pig model is available for investigation. However, until now there has been a lack of incentive for the use of pig as “preferred models”, due to the GM technologies that lag behind the mouse models.

Much of our knowledge of current human biology has been based on studying a large variety of model species. In order to understand the development, diagnosis, and treatment of human diseases, it is important to have a relevant model species in place. While traditional laboratory model species (e.g. rodents, Drosophila, zebrafish, C. elegans) are informative for determining the function of single genes and proteins, it must be recognized that these model species do not always reflect the complexity of human biology. The domestic pig has become increasingly important as a model species for biomedical research due to its many similarities to humans.

Physiologically, swine are remarkably similar to humans in regard to gastrointestinal anatomy and function, cardiovasculature, metabolic syndrome, and comparative organ size. Pigs also have a much longer lifespan than other commonly used animal models, giving researchers the opportunity for longer term studies. Additionally, although its unique evolutionary background places it distinct from primates and rodents, transcriptomic analysis has determined that the pig has higher sequence conservation to the human than the mouse does. This similarity to the human genome is also true for protein coding sequences. Combined, these characteristics make pigs a uniquely suitable model for applications of biotechnology and disease modeling for humans, especially as a bridge between traditional rodent models and nonhuman primates.

### Applications

The presently disclosed techniques can be used to expand the number of progeny that can be generated from any desired donor. The techniques can be used, for example, to facilitate animal breeding. Animals having certain desired traits or characteristics, such as disease resistance, improved fertility and production traits, performance traits, or meat quality traits have long been desired. Traditional breeding processes are capable of producing animals with some specifically desired traits, but these traits are often too time-consuming, costly, and unreliable to develop.

In one aspect, the donor cells are from an elite animal. By “elite animal” as used herein is meant an animal that is highly valuable in terms of genetic traits in productivity, reproduction, disease resistance, or the like. The elite animal may be a sire or a dam. Given that the germline of the chimeric animal, and thus the progeny produced therefrom, are exclusively derived from the donor, the number of offspring which may be produced by a small selection of the best quality parent animals can be vastly increased. Thus, multiplication of livestock animals, e.g., porcine or bovine, with proven genetic superiority or other desirable traits is possible. The technique ensures that large numbers of animals derived from a particular high-quality sire or dam donor can be produced for use in breeding. The obtained chimeric animal can be bred directly, whether by natural mating, artificial insemination, or by in vitro fertilization (IVF) and/or other artificial reproductive technologies.

In another aspect, the donor cells may be obtained from an animal or animal line that is difficult to breed or otherwise maintain. A difficult to breed line may include, for example, an animal that is transgenic, immunodeficient, or lacking one or more functional genes (a knock-out animal). Many such animals are difficult to obtain in large number for use in experiments due to this poor breeding performance.

Multiplication of immunodeficient animals is particularly useful. In one embodiment, the donor cells are from an animal that is immunodeficient. “Immunodeficient,” includes deficiencies in one or more aspects of an animal's native, or endogenous, immune system, e.g. the animal is deficient for one or more types of functioning host immune cells, e.g. deficient for B cell number and/or function, T cell number and/or function, NK cell number and/or function, etc. Immunodeficient mouse models are very useful models for immunology, infectious disease, cancer, and stem cell biology but many are inherently poor breeders.

The obtained chimeric animals may have an essentially normal phenotype, including satisfactory breeding performance, but will produce progeny comprising the donor genetics. In this way, the animals with poor breeding performance are maintained and additional animals can be easily generated. This will result in many progeny derived from the donor in a short period and enables to ability to breed sufficient numbers of the experimental animals.

### EMBODIMENTS

The following numbered embodiments also form part of the present disclosure:
1. A method for producing a chimeric embryo with donor-derived germ cells, the method comprising: providing a host embryo comprising an inactivated primordial germ cell (PGC) specification gene; and complementing the host embryo with donor cells to yield the chimeric embryo, wherein the germ cells of the chimeric embryo are exclusively derived from the donor.
2. The method of embodiment 1, wherein the inactivated PGC specification gene is PRDM14.
3. The method of embodiment 1, wherein the inactivated PGC specification gene is PRDM1, SALL4, IFITM1, DPPA3, DDX4, KITLG, DAZL, DND1, PRMT5, NANOG, AICDA, or TIAL1.
4. The method of any one of embodiments 1-3, wherein the host embryo is complemented at the blastocyst stage.
5. The method of any one of embodiments 1-3, wherein the host embryo is complemented at the 4-cell to 8-cell stage.
6. The method of any one of embodiments 1-3, wherein the host embryo is complemented at the 4-cell stage, 6-cell stage, or 8-cell stage.
7. The method of any one of embodiments 1-6, wherein the donor cells comprise one or more pluripotent cells.
8. The method of embodiment 7, wherein the one or more pluripotent cells comprise embryonic stem cells or induced pluripotent stem cells.
9. The method of embodiment 7, wherein the one or more pluripotent cells comprise a blastomere of a 4-cell stage donor embryo.
10. The method of any one of embodiments 1-9, wherein the animal is non-human.
11. The method of any one of embodiments 1-10, wherein the animal is a mouse.
12. The method of any one of embodiments 1-10, wherein the animal is an ungulate, optionally wherein the animal is a ruminant animal.
13. The method of any one of embodiments 1-10, wherein the animal is a pig or cattle.
14. The method of any one of embodiments 1-13, wherein the inactivation of the PGC specification gene is accomplished by gene editing.
15. The method of embodiment 14, wherein the gene editing comprises use of a TALEN, a zinc finger nuclease, or RNA-guided CRISPR-Cas.
16. The method of any one of embodiments 1-15, wherein the inactivation of the PGC specification gene is accomplished by injecting a zygote with a Cas protein and a guide RNA that targets the PGC specification gene.
17. The method of any one of embodiments 1-16, wherein the donor cells are from an elite animal.
18. The method of any one of embodiments 1-16, wherein the donor cells are from an animal with poor breeding performance.
19. A chimeric embryo produced by the method of any one of embodiments 1-18.
20. The method of any one of embodiments 1-18, further comprising:
transferring the chimeric embryo into a recipient female animal; and allowing the transferred chimeric embryo to develop to term as a chimeric animal.
21. The method of embodiment 20, further comprising: collecting semen from the chimeric animal.
22. The method of embodiment 20, further comprising:
breeding the chimeric animal with a second animal to produce one or more progeny animals.
23. The method of embodiment 22, wherein the breeding comprises natural mating, artificial insemination, or in vitro fertilization.
24. A method for producing a chimeric animal with donor-derived germ cells by blastocyst complementation, the method comprising:
injecting a zygote with a Cas protein and a guide RNA that targets the PRDM14 gene or the PRDM1 gene and allowing the zygote to develop into a blastocyst;
complementing the blastocyst with embryonic stem cells from a donor to yield a chimeric blastocyst, and
transferring the chimeric blastocyst to the uterus of a female recipient animal and allowing a chimeric animal to develop, wherein the chimeric animal comprises germ cells exclusively derived from the donor.
25. A method for producing a chimeric animal with donor-derived germ cells by embryo-embryo aggregation, the method comprising:
injecting a zygote with a Cas protein and a guide RNA that targets the PRDM14 gene or the PRDM1 gene and allowing the zygote to develop into a 4-cell to 8-cell stage embryo; complementing the embryo with a blastomere from a donor 4-cell stage embryo to yield a chimeric embryo; and
transferring the chimeric embryo to the oviduct of a female animal and allowing a chimeric animal to develop, wherein the chimeric animal comprises germ cells exclusively derived from the donor.
26. The method of embodiment 24 or 25, wherein the animal is non-human.
27. The method of embodiment 24 or 25, wherein the animal is a mouse.
28. The method of embodiment 24 or 25, wherein the animal is an ungulate, optionally wherein the animal is a ruminant animal.
29. The method of embodiment 24 or 25, wherein the animal is a pig or cattle.
30. A chimeric animal produced by the method any one of embodiments 24-29.
31. A chimeric embryo comprising host cells and donor cells, wherein the host cells comprise an inactivated primordial germ cell (PGC) specification gene, and wherein the donor cells exclusively contribute to the germ cells of the chimeric embryo.
32. The chimeric embryo of embodiment 31, wherein the inactivated PGC specification gene is PRDM14.
33. The chimeric embryo of embodiment 31, wherein the inactivated PGC specification gene is PRDM1, SALL4, IFITM1, DPPA3, DDX4, KITLG, DAZL, DND1, PRMT5, NANOG, AICDA, or TIAL1.
34. The chimeric embryo of any one of embodiments 31-33, wherein the animal is non-human.
35. The chimeric embryo of any one of embodiments 31-34, wherein the animal is a mouse.
36. The chimeric embryo of any one of embodiments 31-34, wherein the animal is an ungulate, optionally wherein the animal is a ruminant animal.
37. The chimeric embryo of any one of embodiments 31-34, wherein the animal is a pig or cattle.
38. A chimeric animal developed from the chimeric embryo of any one of embodiments 31-37.

All references cited herein are incorporated herein by reference. The examples presented are provided by way of illustration and not meant to limit the scope of the invention.

### EXAMPLES

### Example 1: Direction of Pluripotent Cells Toward a Primordial Germ Cell Fate in Chimeric Mouse Embryos

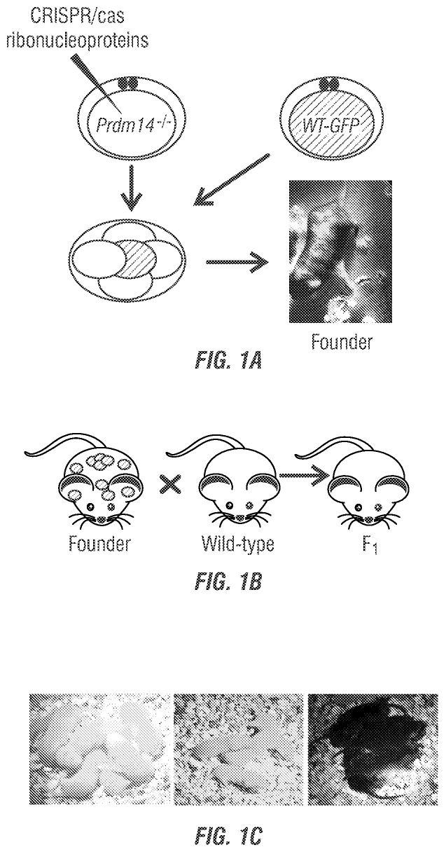

The approach for this example took advantage of the principles for lineage specification; that is, directing a multipotent cell toward a specific cell fate. For this objective, it was necessary to direct the ESC toward a PGC fate so that they would be fully incorporated into the germline. It was hypothesized that by preventing the wild-type embryo from entering the PGC lineage by knocking out the function of Prdm14, the ESC would have uninhibited access to fill the germ cell niche, establishing a chimera that has exclusively donor-derived germ cells (FIG. 1).

### Generation of Prdm14 Chimeric Mice via Embryo-Embryo Aggregation

C57BL/6J (referred to as wildtype (WT); The Jackson Laboratory, Bar Harbor, ME) females were superovulated using intraperitoneal injections of 7.5 IU PMSG (pregnant mare's serum gonadotropin; Sigma, St. Louis, MO) followed by 7.5 IU hCG (human chorionic gonadotropin; Sigma, St. Louis, MO) 46 hours later. WT embryos were collected 10-12 hours post mating with WT males using KSOMaa (potassium simplex optimization medium containing amino acids; Zenith Biotech, Guilford, CT) Evolve media containing HEPES (N-2-hydroxyethylpiperazine-N′-2-ethanesulfonic acid; Zenith Biotech, Guilford, CT).

Zygotes from WT females were moved to FHM handling media (modified KSOM, EMD Millipore; Billerica, MA) and microinjected with a CRISPR guide RNA targeting exon 1 of the Prdm14 gene using 25 ng of Cas9 protein (PNA Bio; Thousand Oaks, CA) and 12.5 ng of guide RNA transcribed in vitro (Ambion MEGAshortscript T7; Austin, TX) and cultured at 37° C. in KSOMaa Evolve (Zenith Biotech; Guilford CT) under 5% oxygen and 5% carbon dioxide.

Embryos from GFP matings were collected on E1.5 at the 2-cell stage. At 2 days post-coitum (dpc), the zona pellucida was removed from GFP embryos and the four blastomeres were separated using a combination of acidic Tyrode's solution (Sigma; St. Louis, MO) and gentle pipetting. One blastomere from the GFP embryo was injected into the 4-cell stage Prdm14 CRISPR-injected non-GFP embryo. Reconstituted embryos were incubated in 150 mg/mL phytohemagglutinin PHA-L (Sigma; St. Louis, MO) for 20 minutes and returned to culture. Embryos were cultured overnight and transferred into the oviduct of day 0.5 pseudopregnant CD1 (Charles River Laboratories; Frederick, MD) females as described below and allowed to go to term.

### Generation of Prdm14 Chimeric Mice Via Blastocyst Complementation (Stem Cell Injection)

C57BL/6J-GFP (GFP: green fluorescent protein; The Jackson Laboratory, Bar Harbor, ME) females were superovulated using intraperitoneal injections of 7.5 IU PMSG followed by 7.5 IU hCG 48 hours later. GFP cumulus-oocyte complexes were collected 14-16 hours post-hCG and placed into a 200 μL in vitro fertilization (IVF) drop of high calcium HTF medium (human tubal fluid) containing 0.25 mM reduced glutathione (Sigma; St. Louis, MO). Table 2 shows the composition of high calcium HTF medium.

Cauda epididymides from ≥8-week old C57BL/6J-GFP males were dissected and gently squeezed to release spermatozoa from the epididymides. Spermatozoa were incubated in TYH (modified Kregs-Ringer bicarvonate medium) containing MBCD (methyl-β-cyclodextrin) at 37° C. under 5% oxygen and 5% carbon dioxide. Table 3 shows the composition of sperm incubation medium (TYH+MBCD). After 1 hour of incubation, 3-5 μL of sperm from the edge of the medium drop were collected and transferred to the IVF drops containing the fresh cumulus-oocyte complexes. Fertilization dishes were then incubated at 37° C. under 5% oxygen and 5% carbon dioxide for 3.25-4 hours.

Presumptive zygotes were microinjected with a CRISPR guide targeting exon 1 of the Prdm14 gene using 25 ng of Cas9 protein (PNA Bio; Thousand Oaks, CA) and 12.5 ng of guide RNA and cultured at 37° C. in KSOMaa Evolve (Zenith Biotech; Guilford, CT) under 5% oxygen and 5% carbon dioxide until blastocyst stage 4 days later. At blastocyst stage, 10-12 embryonic stem cells (R1 control or Cwc15^{-/-} AD7 clone experimental cell line) were injected into the blastocoele of each GFP blastocyst. These blastocysts were transferred into the uterus of day 2.5 pseudopregnant CD1 (Charles River Laboratories; Frederick, MD) females and pregnancies were allowed to go to term.

TABLE 2

| Reagent Name | mg/100 ml |
|---|---|
| NaCl | 593.8 |
| KCl | 35.0 |
| MgSO₄•7H₂O | 4.9 |
| KH₂PO₄ | 5.4 |
| CaCl₂•2H₂O | 75.5 |
| NaHCO₃ | 210.0 |
| Glucose | 50.0 |
| Na-lactate (ml) | 0.34 |
| Na-Pyruvate | 3.7 |
| Penicillin G | 7.5 |
| Streptomycin | 5.0 |
| BSA (Fraction V, Fatty Acid-Free) | 400.0 |
| Phenol Red (0.5% solution) | 0.04 (ml) |

TABLE 3

| Reagent Name | mg/100 ml |
|---|---|
| NaCl | 697.6 |
| KCl | 35.6 |
| MgSO₄•7H₂O | 29.3 |
| KH₂PO₄ | 16.2 |
| NaHCO₃ | 210.6 |
| Na-Pyruvate | 5.5 |
| Glucose | 100.0 |
| CaCl₂•2H₂O | 25.1 |
| Methyl-β-cyclodextrin | 98.3 |
| Penicillin G | 7.5 |
| Streptomycin | 5.0 |
| Polyvinylalcohol | 100.0 |

### Stem Cell Culture

Mouse stem cells for blastocyst injection were cultured in a standard embryonic stem cell culture consisting of 80% DMEM/F-12 (Dulbecco's Modified Eagle Medium; Gibco, Grand Island, NY), 20% fetal calf serum (Atlanta Biologicals; Flowery Branch, GA), 2 mM L-alanyl-L-glutamine dipeptide (Gibco; Grand Island, NY), 0.1 mM non-essential amino acids (Gibco; Grand Island, NY) 1 mM sodium pyruvate (HyClone; Pittsburgh, PA), 0.02 mM β-mercaptoethanol (Gibco; Grand Island, NY), and 1000 U/ml LIF (Leukemia Inhibitory Factor; EMID Millipore, Billerica, MA). Stem cells were passaged every 2-3 days using 0.25% trypsin-EDTA (ethylenediaminetetraacetic acid; Gibco, Grand Island, NY).

Embryo Transfer into Recipient Females

Embryo transfer was performed on either day 0.5 or day 2.5 pseudopregnant CD1 (Charles River Laboratories; Frederick, MD) females, depending on the stage of embryo development. Embryos up to morula stage were transferred into the oviduct of day 0.5 pseudopregnant females and embryos at blastocyst stage were transferred into the uterine horn of day 2.5 pseudopregnant females.

Recipients were weighed using a scale (Sartorius BP610, Sigma; St. Louis, MO) to determine dosage for buprenorphine analgesic (Par Pharmaceuticals; Spring Valley, NY). Buprenorphine was administered subcutaneously at 0.1 mg/kg of body weight. The animal was then moved to the induction chamber of the SomnoSuite Small Animal Anesthesia System (Kent Scientific; Torrington, CT) and induced at a flow rate of 250 mL/min and a concentration of 3.0% isoflurane (VetOne; Boise, Idaho) until the mouse was completely limp. After induction, the mouse was moved to a warming plate and a nose cone placed over its nose. The flow rate was reduced to 200 mL/min with a concentration of 2.0-2.2% isoflurane for the remainder of the procedure. The area just below the distal end of the rib cage down to the top of the knee was shaved on either side of the mouse. The shaved area extended from the dorsal-ventral boundary to the spine. The shaved surgical area was then treated with betadine using a circular scrubbing motion from the central area to the outer edge, and then rinsed with 70% ethanol in the same manner. Eye gel (CLC Medica; Waterdown, ON, Canada) was placed onto the eyes of the animal to reduce drying during the procedure.

Using a stereomicroscope, an initial incision was made roughly ⅓ of the way distally from the ribcage and ⅓ of the way ventrally from the spine. A second incision through the fat and muscle layer was made and the ovarian fat pad located. The ovary and cranial end of the uterine horn was pulled outside the body cavity and placed onto sterile gauze. For oviductal transfer, a small cut was made to the oviduct cranial to the swollen ampulla and 10-20 embryos were transferred using a glass pipette, as well as an air bubble to prevent backflow of embryos out of the oviduct. For uterine transfer, a hole was made in the cranial end of the uterine horn using a 26-gauge needle and 10-15 blastocyst transferred. Once the transfer was complete, the reproductive tract was guided back into the body cavity. The muscle incision was closed using one Halsted suture (Ethicon; Cincinnati, OH) and skin stapled (Roboz; Gaithersburg, MD).

After surgery, mice were placed in a new cage that was pre-warmed at 37° C. until the animal recovered and moved freely. Staples were removed after 10 days and the mice were weighed to determine pregnancy status.

Production of Offspring from Chimeric Animals

To confirm the phenotype of founder chimeras, all founder mice were grown to puberty (5-8 weeks) and then bred with WT animals (in case of embryo-embryo aggregation founders) or with GFP animals (in case of blastocyst complementation founders). These pregnancies were taken to term to generate F1 progeny. The resulting offspring were analyzed for GFP expression. All GFP animals were photographed using a 500 nm emission filter with the NIGHTSEA™ dual fluorescent protein excitation light (Electron Microscopy Sciences; Hatfield, PA) 2-5 days after birth, and WT pups of the same age were photographed as controls under the same lighting conditions. Where time did not permit pregnancies to go to term, females were sacrificed at E1.5 and 2-cell embryos were flushed from the oviducts and cultured in KSOMaa Evolve (Zenith Biotech; Guilford, CT—check location) at 37° C. under 5% oxygen and 5% carbon dioxide until blastocyst stage when they were imaged under a microscope to determine GFP status.

Aggregation of GFP Blastomere with PRDM14^{-/-} null embryos Can Generate Chimeric Mice

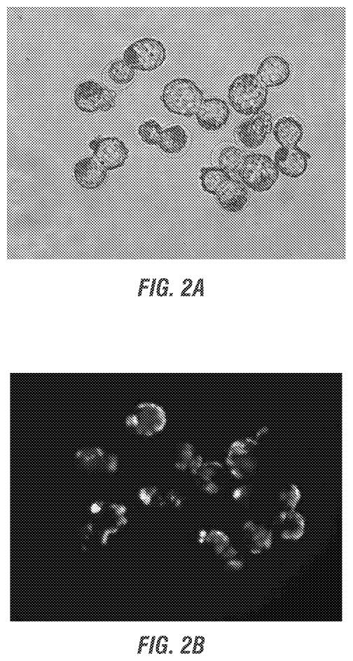

Blastomeres from GFP embryos were aggregated with putative Prdm14^{-/-} mouse embryos. Prior to performing an embryo transfer, embryos were cultured to the blastocyst stage and analyzed for GFP expression. Every embryo showed chimeric GFP expression, indicating that the aggregation was successful and the embryos were capable of developing to the blastocyst stage (FIG. 2).

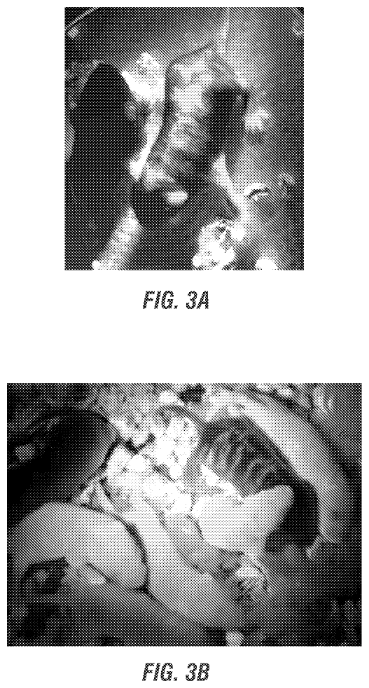

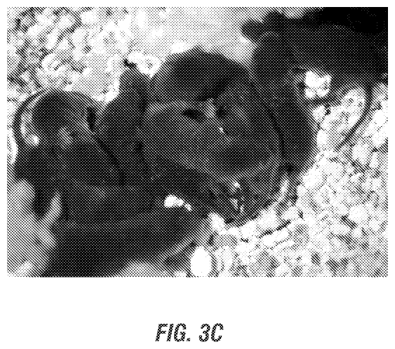

For embryo transfer, the aggregated embryos were transferred the day after aggregation (8-cell to morula stage) to 0.5 dpc pseudopregnant females and pups were born approximately 20 days later. Upon birth, animals were analyzed for GFP expression. From three litters 2 true chimeras were generated, demonstrating that the aggregation was successful due to the appearance of patchy expression of GFP on the individual (FIG. 3). Seven other animals were generated that were 100% GFP-expressing, indicating that the donor blastomere was responsible for giving rise to the entire mouse, as seen from GFP expression externally. Table 4 is a summary of generation of chimeric founders (F0) from embryo aggregations.

TABLE 4

| | | | Percentage |
|---|---|---|---|
| Replicate | # Chimeras | Litter Size | Chimeric |
| 1 | 1 | 7 | 14.29% |
| 2 | 1 | 9 | 11.11% |
| Total | 2 | 16 | 12.50% |

Founder Chimeric Individuals of Embryo-Embryo Aggregations have Germline Originating from Donor Blastomeres

The founder animals from embryo-embryo aggregations were then raised until puberty (5-8 weeks), when they were mated with WT individuals. This mating was performed to determine if the germ cells of the founder animals arose completely from the GFP donor blastomere as expected. If the germ cells were all generated from the GFP donor embryo, then after mating, subsequent offspring should all be GFP positive, indicating 100% occupation of the germline by the GFP embryo donor lineage. Each founder individual (including those founders assessed to be 100% GFP) was mated to produce at least 1 litter for analysis. Across all litters, every F1 pup born was 100% GFP positive, indicating that all germ cells from the chimeric parent were of donor origin. Table 5 is a summary of GFP offspring from founder chimeric individuals (F₁).

TABLE 5

| Parent ID | % Chimerism | # GFP Pups | # Total Pups | Percentage |
|---|---|---|---|---|
| 1 | 50 | 16 | 16 | 100 |
| 2 | >90 | 19 | 19 | 100 |
| 3 | >90 | 8 | 8 | 100 |
| 4 | >90 | 8 | 8 | 100 |
| 5 | >90 | 9 | 9 | 100 |
| 6 | >90 | 8 | 8 | 100 |

Blastocyst Complementation Generates Chimeras from R1 ESC

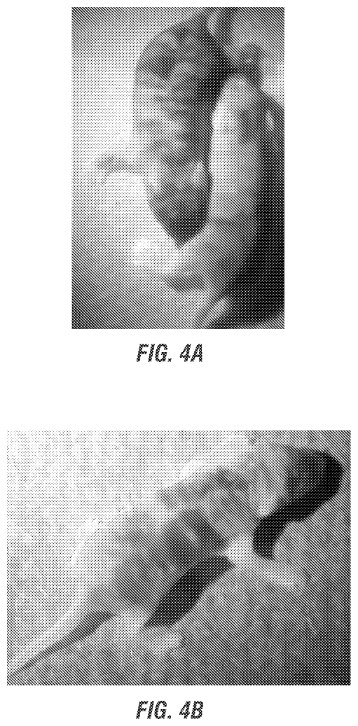

After delivery by the surrogate recipient mother, 3 founder animals were generated from embryos that were injected with the robust R1 control stem cell line. In this experiment, the host embryo was GFP-expressing while the donor stem cells were not. This was the opposite expression pattern as the previous embryo-embryo aggregation experiment. However, founder animals still retained the classic look of a chimera, with a patchy coat including GFP positive and GFP negative areas (FIG. 4).

R1 Founder Chimeras have Germline Originating from ESC Background

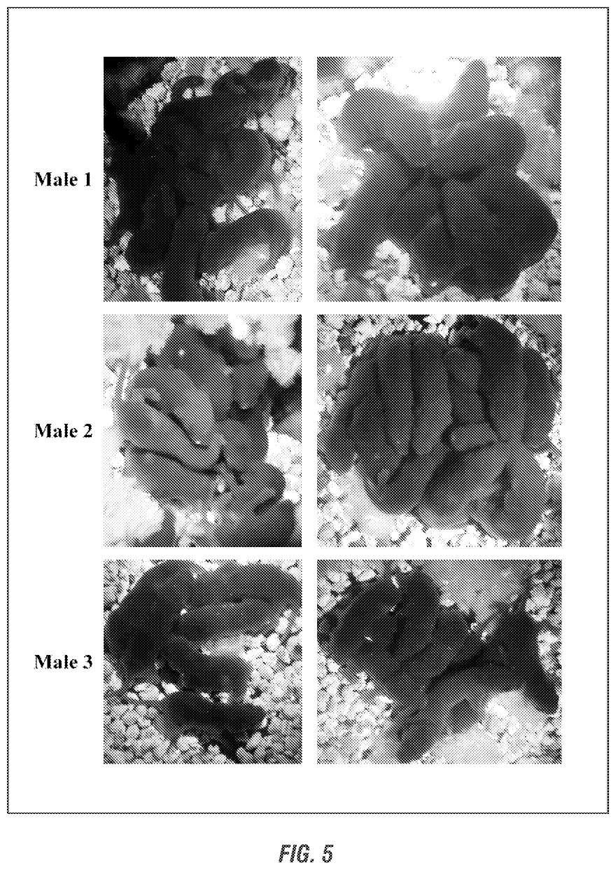

The founder animals from blastocyst complementations using R1 control ESC were raised to puberty and then mated to WT mice. In this experiment, the donor R1 stem cells did not express GFP but were injected into a host embryo that did express GFP. Therefore, if the R1 stem cells contributed to the germ cell lineage, they would not express GFP, and resulting offspring from mating to a WT individual should also be GFP negative. Each founder animal was mated to produce at least 3 litters. Each litter produced by a founder animal consisted of pups that were all GFP-negative, indicating that the germ cells from their founder parent were 100% from R1 stem cell origin (FIG. 5). Table 6 is a summary of mating results of founder R1 chimeras with WT females.

TABLE 6

| Mouse ID | Sex | Mate | Litter Birthdate | Litter Size | Germline Origin |
|---|---|---|---|---|---|
| 1R1 | M | C57BL/6J | Jul. 11, 2017 | 10 | R1 ESC |
| | | C57BL/6J | Aug. 4, 2017 | 9 | |
| | | CD1 | Aug. 9, 2017 | 8 | |
| | | CD1 | Sep. 24, 2017 | 7 | |
| 2R1 | M | C57BL/6J | Jul. 10, 2017 | 11 | R1 ESC |
| | | C57BL/6J | Aug. 3, 2017 | 5 | |
| | | CD1 | Aug. 8, 2017 | 3 | |
| | | C57BL/6J | Aug. 28, 2017 | 11 | |
| 3R1 | M | C57BL/6J | Sep. 19, 2017 | 10 | R1 ESC |
| | | C57BL/6J | Sep. 26, 2017 | 6 | |

Cwc15^{-/-} Founder Animals Show Lack of Chimerism and Cwc15^{-/-} ESC do not Contribute to the Germline

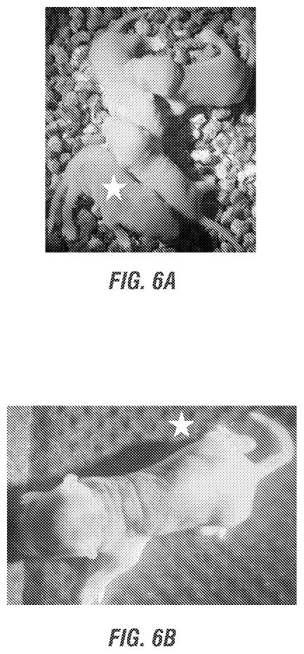

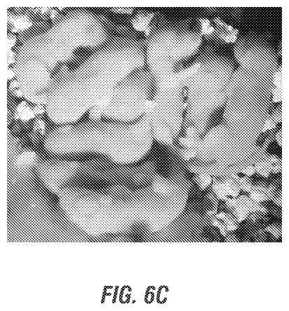

Blastocyst complementation was also performed using ESC that were mutant for Cwc15 (Cwc15^{-/-}). This stem cell line was chosen because previous experience in our lab with this line generated chimeras quite easily but none of these were germline chimeras. It was therefore decided that the Cwc15^{-/-} line would be an ideal candidate to determine if previously germline-incompetent ESC could be pushed toward occupation of the germline once endogenous PGC competition was removed. Unfortunately, the blastocyst complementation approach yielded only 1 visible chimera, which died at 12 weeks of age prior to siring a litter. The other founder animals showed low contribution by ESC, and did not yield any visible chimeras. Instead, all pups produced from this experiment were almost entirely GFP positive, indicating that the offspring were derived solely from the host embryo, with little to no contribution by the Cwc15^{-/-} ESC. A total of 5 animals were produced, with only 2 showing any sign of contribution by the stem cells (≤5% GFP negative). One of the limited chimeric animals however was phenotypically female, indicating that the ESC did not contribute to her reproductive system, as the ESC were karyotypically XY, and therefore should only give rise to male offspring if incorporated into the reproductive tract during development. Images of the founder animals are shown in FIG. 6. Table 7 is a summary of mating results of founder CWC15^{-/-} chimeras with WT mates. An asterisk indicates the one visible chimera that died at 12 weeks of age.

TABLE 7

| Mouse ID | Sex | Mate | Litter Birthdate | Litter Size | Germline Origin |
|---|---|---|---|---|---|
| 1CWC | F | C57BL/6J | Oct. 6, 2017 | 3 | Host Embryo |
| | | | Nov. 16, 2017 | 4 | Host Embryo |
| 2CWC | M | C57BL/6J | Dec. 2, 2017 | 7 | Host Embryo |
| 3CWC | M | C57BL/6J | Oct. 17, 2017 | 8 | Host Embryo |
| | | | Nov. 19, 2017 | 8 | Host Embryo |
| 4CWC | F | C57BL/6J | Oct. 2, 2017 | 9 | Host Embryo |
| | | | Nov. 19, 2017 | 4 | Host Embryo |
| 5CWC* | M | C57BL/6J | N/A | N/A | N/A |

### Discussion

In this example, we sought to determine if ESC could be preferentially biased toward a germline fate by eliminating endogenous PGC in the host embryo. To accomplish this, Prdm14 was knocked out in a host embryo that was then aggregated with either another WT embryo or with ESC. Previously, our lab obtained results from a chimera experiment in which it was possible to generate chimeras from the Cwc15^{-/-} ESC line. However, none of the chimeras produced showed germline transmission, as offspring from these chimeras were all wild-type. This led to an important experimental question: were the stem cells simply unable to contribute to the germline and therefore not fully competent, or were the stem cells being outcompeted by the endogenous germ cell program? As evidenced by this example, the answer may comprise a bit of both explanations.

It is already known that stem cells are able to rescue a knockout phenotype when introduced into a mutant blastocyst. In fact, this has been reported numerous times with researchers targeting genes important for whole organ generation, such as Pdx1 for the pancreas and Nkx2.5 for the heart. So far, blastocyst complementation (the process of injecting stem cells into a genetically modified embryo) has been used in the mouse to rescue the function of Runx2 (Chubb, Oh et al. 2017), Nkx2.5 (Sturzu, Rajarajan et al. 2015), Oct4 (Le Bin, Munoz-Descalzo et al. 2014), Sall1 (Usui, Kobayashi et al. 2012), Rag2 (Chen, Lansford et al. 1993), Pdx1 (Kobayashi, Yamaguchi et al. 2010, Kobayashi, Kato-Itoh et al. 2015), and Id1/2/3 (Fraidenraich, Stillwell et al. 2004), among countless others. However, blastocyst complementation and the rescue of the phenotype has not been shown in the reproductive system to date. This example is the first step in generating a rescue phenotype for primordial germ cells via blastocyst complementation.

In addition to providing evidence that ESC can rescue a knockout PGC phenotype, this study provides a foundation for generating chimeras from ESC that were previously unable to show germline transmission. While the robust R1 ESC line generated chimeras easily, the Cwc15^{-/-} line did not, which was unexpected as chimeras had been previously produced by our group. This may be explained by a change in experimental methods between the two attempts. Previously, our chimeras were generated by collection of embryos at the blastocyst stage, injection of 10-12 stem cells into the blastocoele, and immediately transferred back into the surrogate mother. Due to the nature of this experiment and the need for CRISPR/Cas9 injection, embryos were cultured from zygote stage to blastocyst stage, a time spanning 4 days. After injection of ESC into the blastocyst, embryos were allowed to recover for 1-2 hours. This combination of experimental conditions could explain the decrease in developmental potential of the whole embryos. The use of blastocyst complementation may also explain the low derivation of pups, as the embryos were manipulated twice.

In addition to not generating chimeras efficiently, of those that were produced, the degree of chimerism was quite low and there was little to no contribution by the Cwc15^{-/-} ESC to the whole animal. Despite the low degree of chimerism seen externally, it was still possible however unlikely that the germ cell population was generated by the Cwc15^{-/-} cells so the founder animals were mated to determine the status of the germ cell lineage. As expected, offspring generated from these matings were entirely GFP positive, indicating a lack of contribution to the germline by the Cwc15^{-/-} ESC, as in this chimera experiment the host embryo had GFP expression while the donor ESC did not.

Despite the lack of contribution to the germline by the Cwc15^{-/-} ESC, it may still be possible to use this technique to generate germline competent chimeras. Additional ESC lines should be tested to generate animals with a higher degree of chimerism than were produced in this example. Producing founders with a high degree of chimerism is a critical first step in determining if ESC can contribute to the germline. Without that criterion met, this study did not have a good baseline from which to determine germline competence of the Cwc15^{-/-} ESC.

The present chimeric mouse study in which embryonic stem cells were directed toward the germ cell lineage provides another approach to generating germline chimeras. The mouse ESC field has been plagued with cells that are either developmentally incompetent for PGC or are outcompeted by the endogenous PGC. This study provides a useful mechanism to overcome the germline transmission barrier so that the field can continue to move forward and characterize the function of genes, the modeling of human disease, and the biology of reproduction.

### Example 2: Expression of Germline-Specific Genes in the Early Porcine Embryo

Because of its role in both PGC specification and maintenance of pluripotency in the mouse, and its expression in human ESC, investigation of the role of PRDM14 in a non-rodent “bridge model” is warranted. While the pig genome has been sequenced, not all genes have been annotated and many annotated genes have yet to have their functions described. In order to determine the function of PRDM14 in the pig, it was necessary to first determine its normal expression profile at similar developmental time points and locations as are found in the mouse.

Along with determining the expression of PRDM14 at varying time points in early pig development, several other genes of interest were also chosen for analysis. There were 4 groupings of genes to consider: PGC-related genes (PRDM1 and TFAP2C), genes involved in epigenetic modifications (CARM1, TET1, and TET2), pluripotency genes (POU5F1, SOX2, NANOG, and ESRRB), and germ cell markers (DDX4, DAZL, and STRA8). These groups of genes were chosen based on known information about their function in the mouse system, as well as interest in how genes that are associated with PRDM14 may be functioning at these developmental time points.

### Collection of In Vivo Embryos

A cohort of gilts were synchronized by oral administration of progesterone analog REGU-MATE® (0.22% altrenogest solution, 2.2 mg/mL) starting from day 15 after a gilt showed behavioral heat. Animals were given 22 mg (10 mL) REGU-MATE® once daily via a drench gun for a minimum of 6 days prior to withdrawal. Approximately 5-7 days after REGU-MATE® withdrawal, gilts in standing estrus were bred 2-3 times via artificial insemination. Semen for AI was provided by Genus PIC in individual doses. Animals were sacrificed based on the stage of embryo desired, as shown in Table 8.

TABLE 8

| Developmental Stage | Day euthanized after breeding |
|---|---|
| Zygote | 1 |
| 2-cell | 2 |
| 4-cell | 3 |
| Morula | 4 |
| Blastocyst | 6 |
| e26 | 26 |

Reproductive tracts were removed from the females and flushed via 18 gauge needle and syringe using 30-35 mL warmed Dulbecco's Modified Eagle Medium (DMEM, Gibco; Grand Island, NY). Depending upon the stage of development, either the oviduct (zygote and 2-cell), uterine horns (blastocyst), or both (4-cell and morula) were flushed. For embryonic day (E) 26 samples, fetuses were carefully removed from the reproductive tract inside a laminar flow hood and the gonads were dissected for collection. All embryos were then immediately collected for RNA. Gonads from E26 fetuses were snap frozen in liquid nitrogen prior to RNA extraction.

RNA Collection and cDNA Synthesis

RNA from various stage embryos was collected using the DYNABEADS™ mRNA Purification kit according to manufacturer's instructions (ThermoFisher; Waltham, MA) into a final volume of 10 μL. First, the zona pellucida of each embryo was removed using acidic Tyrode's solution (Sigma; St. Louis, MO) for 2-3 minutes until the zona was completely dissolved. For embryos from developmental stages zygote through morula, 2-3 embryos were pooled for RNA collection to ensure enough RNA to process for PCR. Blastocysts were harvested individually based on evidence from previous publications (Park, Jeong et al. 2012). For E26 gonad samples, RNA was extracted using the RNeasy Mini Kit according to manufacturer's instructions (Qiagen; Hilden, Germany). Briefly, the sample was ground using a tenbroeck homogenizer that was pre-chilled in liquid nitrogen. The sample was then passed through a 20-gauge needle before proceeding with the RNeasy kit, and eluted in a final volume of 30 μL.

cDNA was then synthesized via the oligo(dT) method using the SuperScript™ IV First-Strand Synthesis System according to manufacturer's instructions (ThermoFisher, Waltham, MA). Using a final volume of 20 μL, synthesis was carried out at 50° C. for 15 minutes and the reverse transcription reaction was terminated by incubating at 80° C. for 10 minutes.

### Droplet Digital™ PCR

PCR to identify expression of genes of interest was performed using the Bio-Rad QX200 DROPLET DIGITAL™ PCR system (ddPCR™) according to manufacturer's recommendations (Hercules, CA). In this system, a single PCR reaction is partitioned into thousands of reactions by placement inside of oil droplets which are amplified and quantitated individually. This allows for quantitative analysis of samples with low starting material or copy number, while giving thousands of data points for a single sample. This system also provides absolute measurement of copy number without the need for running standard curves.

For each sample, a 22 μL reaction using ddPCR™ EvaGreen Supermix was loaded into a 96-well plate using the specific primers listed below. The plate was loaded into the QX200™ Automated Droplet Generator (Bio-Rad; Hercules, CA) where the sample was fractionated into 12,000-20,000 individual droplets. After droplet generation, samples were amplified using the C1000 TOUCH™ Thermal Cycler (Bio-Rad; Hercules, CA) using the following conditions: 95° C. for 10 minutes followed by 40 cycles of 94° C. for 30 sec and 58° C. for 1 min, and a final signal stabilization step of 98° C. for 10 minutes. After amplification, the plate was transferred to the QX200™ Droplet Reader for reading and analysis of the droplets using the absolute quantification setting on the machine. Primer sequences used for ddPCR™ are shown in Table 9.

TABLE 9

| Gene | Primer |
|---|---|
| RPS18 | F: 5′-GGCTACCACATCCAAGGAAG-3′ (SEQ ID NO: 79) |
| | R: 5′-TCCAATGGATCCTCGCGGAA-3′ (SEQ ID NO: 80) |
| PRDM14 | F: 5′-GAAGTCAAGACCCACGGAGA-3′ (SEQ ID NO: 81) |
| | R: 5′-AGTTCCCAGCACCTCCTTTT-3′ (SEQ ID NO: 82) |
| PRDM1 | F: 5′-TGTGGGTACGACCTTGGCTG-3′ (SEQ ID NO: 83) |
| | R: 5′-CATATCCGCGTCCTCCATGT-3′ (SEQ ID NO: 84) |
| CARM1 | F: 5′-ACCACACGGACTTCAAGGAC-3′ (SEQ ID NO: 85) |
| | R: 5′-CGTAGATCTTCCTCGCTCCA-3′ (SEQ ID NO: 86) |
| TFAP2C | F: 5′-TCGTGCTCTGACCTTGAAGT-3′ (SEQ ID NO: 87) |
| | R: 5′-CTCTTGCCATCTCCTTGTGC-3′ (SEQ ID NO: 88) |
| TET1 | F: 5′-AGGCGGCCGACCAAAAC-3′ (SEQ ID NO: 89) |
| | R: 5′-GGCACGAGGAACAGAGTCAT-3′ (SEQ ID NO: 90) |
| TET2 | F: 5′-AGCTCACCGAAACACTGAGG-3′ (SEQ ID NO: 91) |
| | R: 5′-CAGGCACAGGTTCTCTCTTCA-3′ (SEQ ID NO: 92) |
| ESRRB | F: 5′-GCCCGTACCTGAGCTTACAG-3′ (SEQ ID NO: 93) |
| | R: 5′-AGGCATGGCGTAGAGTTTGT-3′ (SEQ ID NO: 94) |
| STRA8 | F: 5′-ATGTGGCAAGTTTCCTGGAC-3′ (SEQ ID NO: 95) |
| | R: 5′-GAAACTTCTCCTCGGGCTTT-3′ (SEQ ID NO: 96) |
| DAZL | F: 5′-CCTCCAACCATGATGAATCC-3′ (SEQ ID NO: 97) |
| | R: 5′-ACACAGGCAGCTGATAACCA-3′ (SEQ ID NO: 98) |
| DDX4 | F: 5′-GAGAGGCGGCTTTCAAGATG-3′ (SEQ ID NO: 99) |
| | R: 5′-TAACCACCTCGTCCACTTCC-3′ (SEQ ID NO: 100) |
| SOX2 | F: 5′-CCTACGGGACATGATCAGCA-3′ (SEQ ID NO: 101) |
| | R: 5′-CTCCAGTTCACTGTCCGGC-3′ (SEQ ID NO: 102) |
| NANOG | F: 5′-GGTTTATGGGCCTGAAGAAA-3′ (SEQ ID NO: 103) |
| | R: 5′-GATCCATGGAGGAAGGAAGA-3′ (SEQ ID NO: 104) |
| POU5F1 | F: 5′-GGGTTCTCTTTGGGAAGGTGT-3′ (SEQ ID NO: 105) |
| | R: 5′-TGCCTTGCATATCTCCTGCA-3′ (SEQ ID NO: 106) |

### Statistical Analysis

Transcript copy number for each target gene at each developmental stage was normalized to an internal reference (40S Ribosomal protein S18; RPS18) corresponding to the appropriate developmental stage to correct for differing amounts of starting RNA. The following equation was used for normalization of each target gene: mRNA level=(Transcript copy number)_{target}/(Transcript copy number)_{RPS18}. The data were log 2-transformed prior to analysis by ANOVA using the MIXED models procedure of SAS (SAS Institute; Cary, NC) and differences between the developmental stages were examined using the test of least significant difference (PDIFF). A significance level of p<0.05 was used to determine significance. The data are presented relative to the earliest embryonic stage examined for each gene, which was expected to have the lowest level of expression among developmental stages.

### Pluripotency Genes are Upregulated in the Early Embryo and in the E26 Fetal Gonad

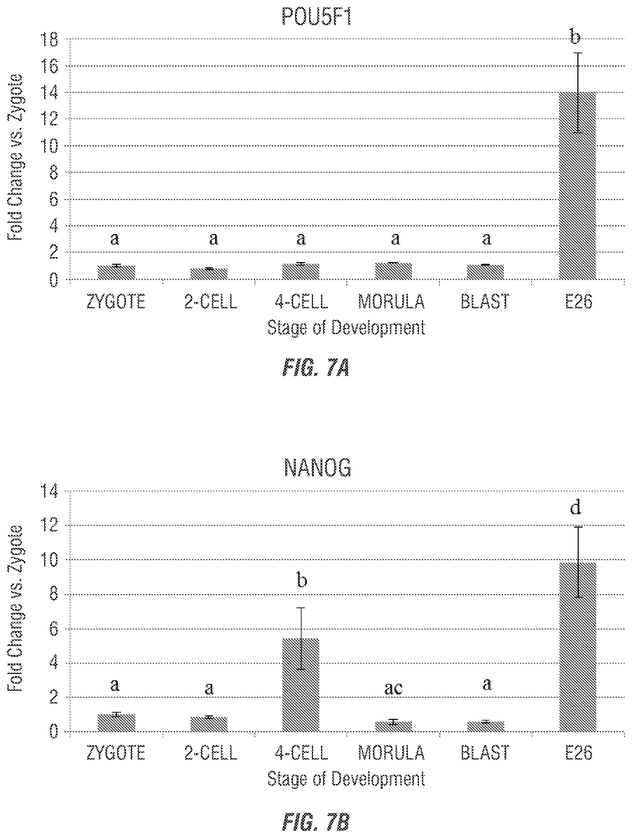

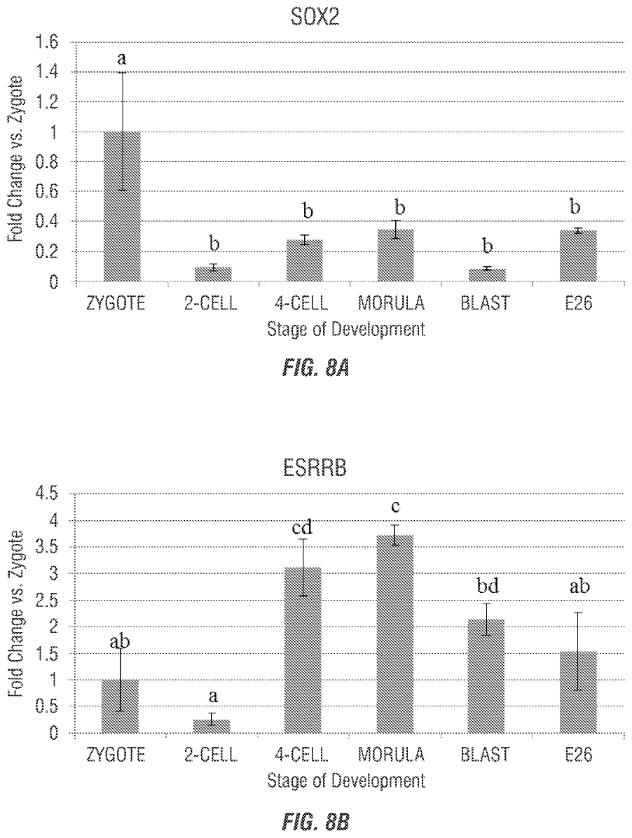

Pluripotency genes POU5F1, SOX2, NANOG, and ESRRB were chosen for inclusion in this study to serve as positive controls for early embryo expression, and to determine if they were also characteristic markers of the PGC population at E26. Until recently, POU5F1 and NANOG were some of the only markers used to identify PGCs during porcine fetal development due to the lack of knowledge regarding PGC signaling and specification pathways. These data show a similar pattern of POU5F1 and NANOG: expression at all stages of development analyzed with higher expression in the fetal gonad than in the preimplantation embryo (FIG. 7). As expected, ESRRB is also upregulated in PGC at E26, as well as in the 4-cell to blastocyst stages of the preimplantation embryo. SOX2 however showed diminished expression throughout development when compared to expression levels at the zygote stage (FIG. 8).

### Primordial Germ Cell-Related Genes Show Little Expression in the Early Embryo

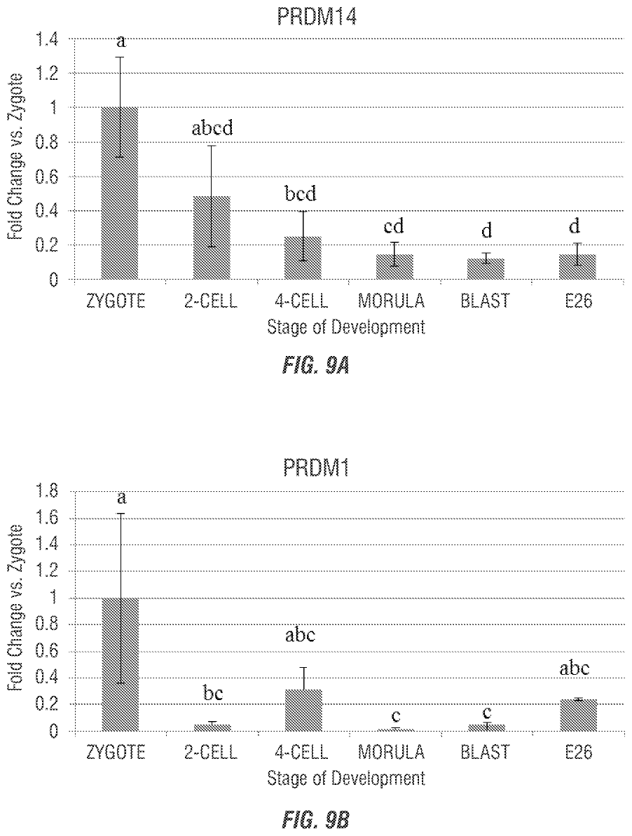

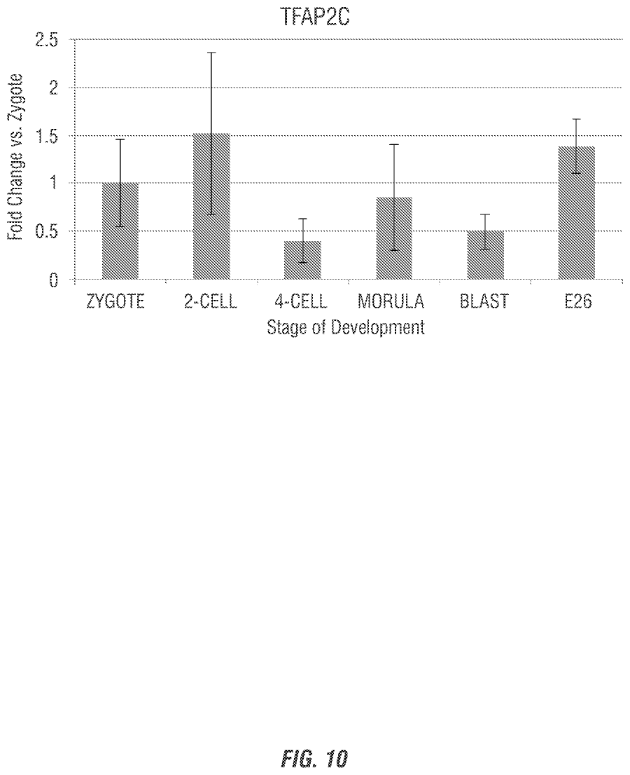

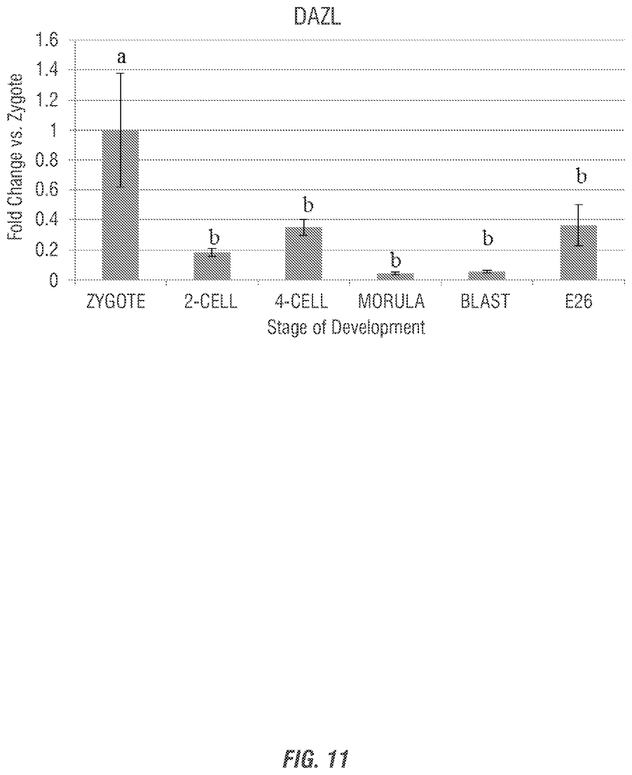

In order to determine if the genes important for germ cell specification in the mouse are also expressed at key time points in the pig, we chose for analysis the three PGC-related genes that are necessary and sufficient for mouse PGC specification: PRDM1, PRDM14, and TFAP2C. PRDM1 and PRDM14 showed differential expression across the 6 stages of development analyzed, with reduced expression as development progresses (FIG. 9). TFAP2C showed no significant pattern of expression across developmental time points (FIG. 10).

### Germ Cell Markers are not Expressed in the Early Embryo

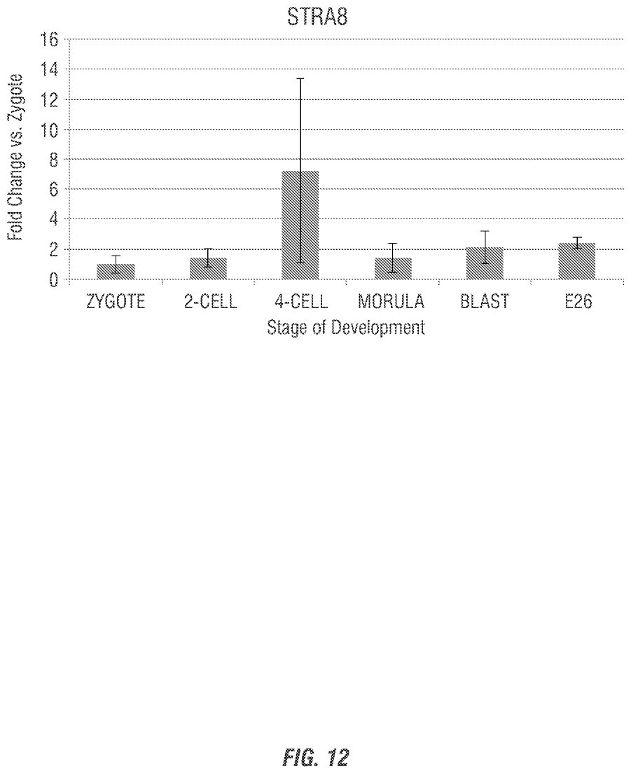

DAZL, VASA, and STRA8 are all markers of the germ cell population. Unlike the other two genes, STRA8 is restricted to the post-natal male lineage. In this experiment, we included germ cell markers in the study to determine if their expression was limited to the germ cell population, or if there was some earlier expression in pluripotent cells of the preimplantation embryo. DAZL and VASA both showed high expression at the zygote stage, with tapering levels as development continued (FIG. 11). DAZL in particular showed an increased level of expression at the E26 time point, indicative of its role in the pre-natal germ cell population. STRA8 transcript levels were low across all time points and this gene did not exhibit any significant trends in expression across time points (FIG. 12).

### Genes Involved in Epigenetic Reprogramming are Upregulated During Periods of Genome-Wide Demethylation

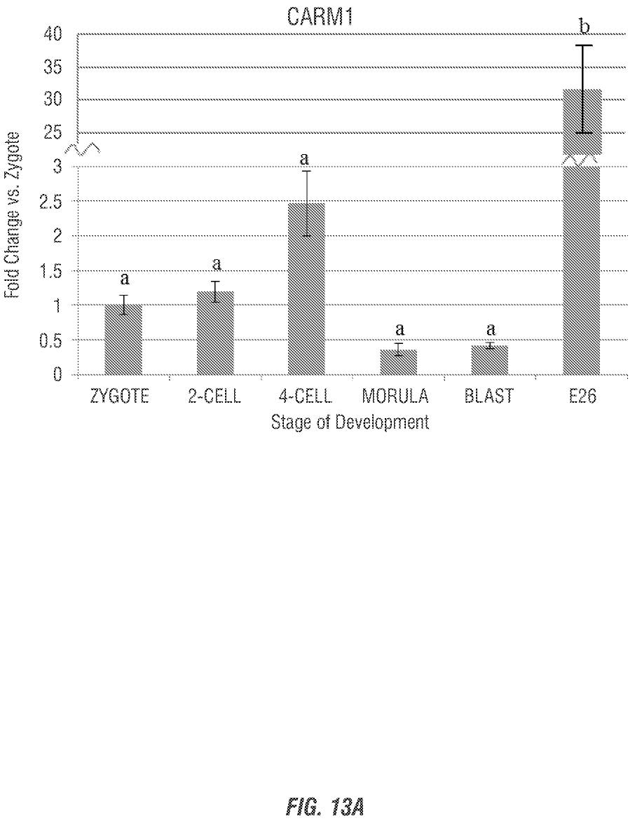

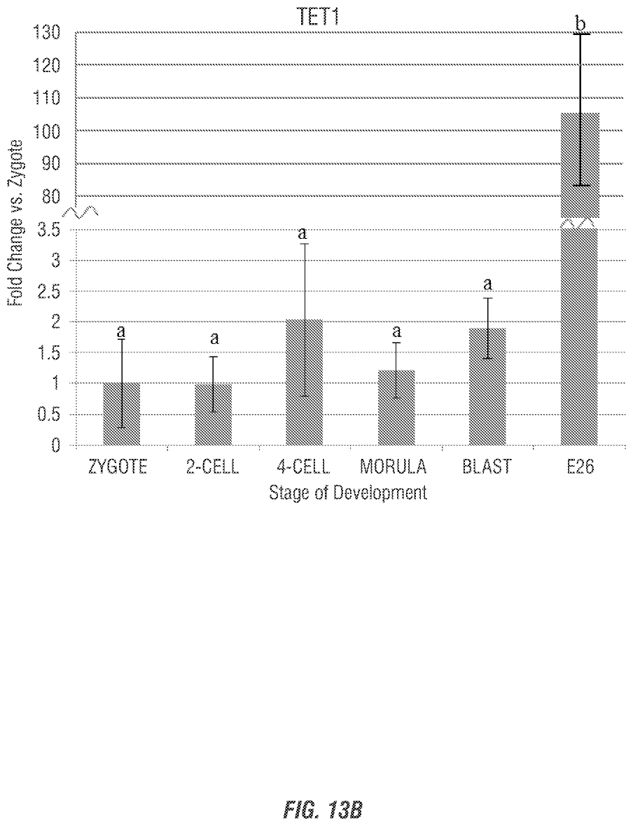

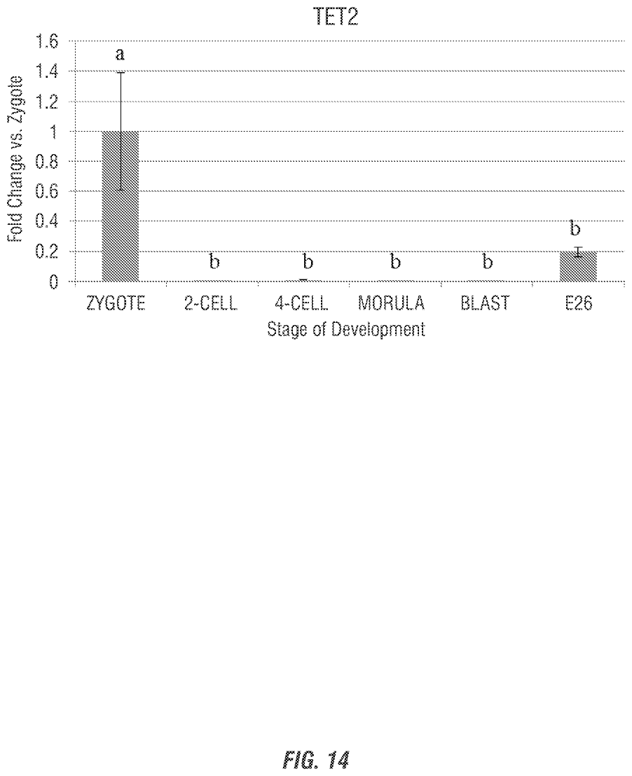

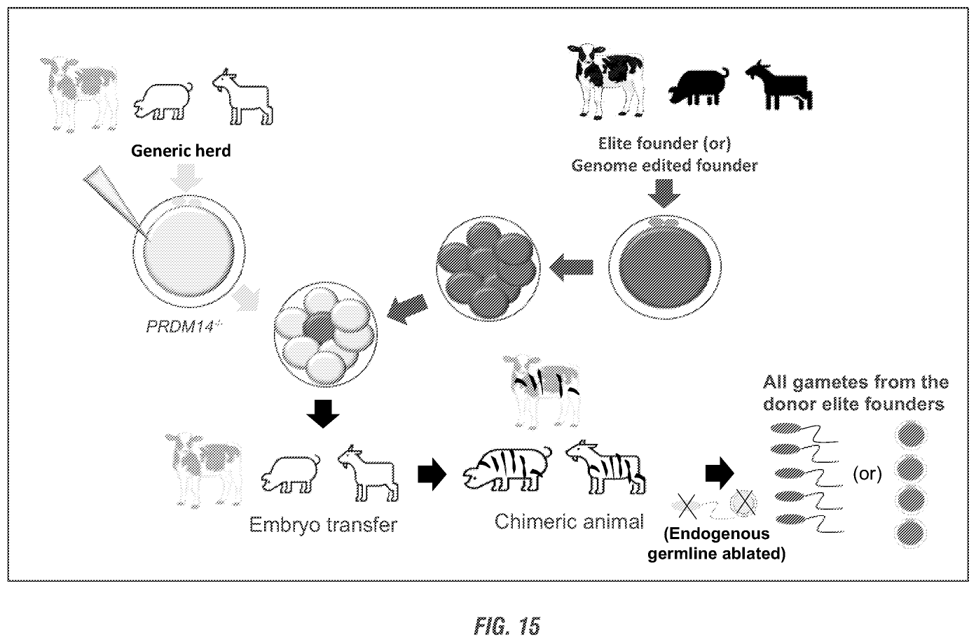

The mammalian embryo undergoes two main rounds of genome-wide DNA methylation reprogramming: in the early preimplantation embryo and in PGC. Therefore, we chose to investigate three factors involved in DNA methylation reprogramming that have also been shown to interact with PRDM14 in other species. Of the genes chosen for analysis, CARM1 and TET1 were both upregulated in E26 gonads (FIG. 13). In contrast, TET2 showed highest expression at the zygote stage (FIG. 14). All three of these genes showed the highest level of expression at one of the two developmental time points where epigenetic marks are being erased genome-wide, indicating a positive correlation with this process.

### Discussion

The above experiment describes for the first time in the porcine system the expression pattern of several PGC, germ cell, and epigenetic markers in the preimplantation embryo. The genes chosen for analysis in this experiment were chosen based on their proposed role within the germ cell program or their association with the major gene of interest, PRDM14.

The pluripotency markers POU5F1 and NANOG showed increasing expression as the embryo continued to develop, with highest expression levels at E26. This is likely due to an increase in the number of cells of the embryo through blastocyst stage, as well as very high expression in the pluripotent PGCs that reached the genital ridge by E26. SOX2 and ESRRB did not have this same trend for increased expression at the E26 stage. It is possible that the lower expression at E26 of these factors is caused by a dilution effect based on the increased number of cells at this stage of development, or lower overall expression as compared to POU5F1 and NANOG. This same effect could be true for several other factors that were expected to be highly expressed in the genital ridge (PRDM14, PRDM1, DAZL, VASA).

The PGC marker PRDM1 showed highest expression at the zygote stage. This indicates that there was little to no expression as development occurred, at least through blastocyst stage. In the pig, PRDM1 has been shown to be expressed in the PGC and prespermatogenesis population of cells (Petkov, Marks et al. 2011, Kakiuchi, Tsuda et al. 2014, Kobayashi, Zhang et al. 2017). Kobayashi et al found that it had higher expression than PRDM14 in the PGC population of both pigs and humans, so it is expected that there would be some expression at E26 in this study. Again, dilution of the effect due to large number of cells in the gonad at this stage may account for the reduced effect seen graphically. In the pig, PRDM1 has 7 transcript variants to examine. The primers used here detected variants X2 and X7, which are two of the three longest transcript variants produced. It is therefore possible that amplification of the other 5 variants would result in some expression in the early porcine embryo. Currently it is unknown which variants are expressed in the germ cell lineage in pigs, because PRDM1 is also expressed in other tissues of the body, as evidenced in the mouse (Mould, Morgan et al. 2015, Ahmed, Elias et al. 2016, Bankoti, Ogawa et al. 2017). However, this same primer set showed amplification of message within the whole fetal gonad, indicating it was appropriate for use in this study.

PRDM14 also had low expression throughout early embryo development and even at E26 (Kobayashi, Zhang et al. 2017). While TFAP2C expression is low in this experiment, TFAP2C may be constitutively expressed and may not be upregulated at any specific time point examined. In the mouse system, Tfap2c is important for specification of the trophectoderm lineage during the morula to blastocyst transition, as well as placental development (Auman, Nottoli et al. 2002, Winger, Huang et al. 2006, Choi, Carey et al. 2012, Cao, Carey et al. 2015). However, this increase in expression in the preimplantation embryo was not seen in this pig study.

Three germ cell markers that identify pre- and post-natal germ cells were included for analysis—DAZL, VASA, and STRA8. Similar to PGC markers, these were chosen because they are indicative of cells in a pluripotent pathway and may therefore have shown expression in the early embryo. Both DAZL and VASA showed highest expression at the zygote stage with little to no expression after that time period. DAZL in particular showed another increase of expression at E26, although this change was not significant. Because these two are markers of post-natal germ cells, it is likely that the high expression at zygote stage is holdover of transcript deposited prior to fertilization, especially since levels drastically decreased following the first cleavage event. The third germ cell marker, STRA8, is considered a meiotic gatekeeper gene as it regulates a germ cell's entry into meiosis (Anderson, Baltus et al. 2008, Feng, Bowles et al. 2014, Wang, Chen et al. 2014). In the early pig embryo, it has very little expression, indicating it is not required for early development.

The last suite of genes chosen for analysis were genes involved in epigenetic reprogramming—CARM1, TET1, AND TET2. Each of these genes has been shown to interact with PRDM14 in the mouse system, as described previously. The ddPCR™ data shown here indicate that they are active in the pig system, with highest expression at one of the two periods of genome-wide epigenetic reprogramming. CARM1 and TET1 both showed exceptionally high expression during the PGC stage of development while TET2 showed higher expression at the zygote stage.

The atypical expression profile of STRA8 at the 4-cell stage along with three other genes that show similar anomalies at that stage (NANOG, PRDM1, DAZL) is of interest. This rise in expression at that single time point could be due to degradation of sample, or to a reduction in overall transcription that would result in a skewed ratio of the gene of interest to the housekeeping gene. In pigs, the maternal to zygotic transition occurs during the 4-8 cell stage (Prather 1993, Lee, Hamm et al. 2014). During this time, the zygotic genome is activated resulting in new transcription from the embryonic genome, and degradation of maternally inherited mRNA transcripts. The 4-cell stage embryos used for analysis in this experiment could have been collected during this critical transition phase, resulting in the differences in expression for these four genes.

This study provides valuable information regarding the expression of key germline-related factors during early pig development. These results clearly show low levels expression for the germ cell-related genes (PRDM14, PRDM1, TFAP2C, DAZL, VASA, STRA8. The low expression of germ cell related genes at E26 and earlier timepoints could be because of the low number of germ cells compared to somatic cells in the gonad, that results in the net dilution of the transcript copy number. Single cell sequencing may be an appropriate mechanism to investigate relative transcript levels in the PGC and germ cells, which is part of an ongoing effort in the laboratory. The data presented in this experiment represent a key first step in describing the normal expression profile of the porcine embryo, as well as delineating the role of PRDM14 at these early developmental stages.

### Example 3: Ablation of PRDM14 in Pigs Results in the Loss of Primordial Germ Cells Similar to Rodent Studies

Our knowledge of mammalian PGC development and gametogenesis comes from studies in mouse. In this example, we generate PRDM14 null porcine embryos that result in fetuses that lack the ability for functional spermatogenesis or oogenesis. When such embryos are complemented with donor embryonic cells, the resultant chimeric founder will have all the germ cells of donor origin (FIG. 15).

As shown in Example 1, feasibility studies have already been performed in the rodent model. Briefly, wild type mouse embryos were injected with CRISPR/Cas ribonucleoproteins targeting Prdm14 into the cytoplasm of the embryo to cause out-of-frame mutations and knockout of Prdm14. At 4-8 cell stage, when the putative Prdm14^{-/-} embryos are aggregated with donor-derived embryonic cells (from GFP transgenic mice) or pluripotent cells (embryonic stem cells), and transferred into estrus synchronized surrogate recipient animals, the resulting offspring is chimeric and contains both donor and recipient somatic cells; however, the germline is contributed exclusively by the donor cells. Aggregation of the Prdm14^{-/-} embryos with a blastomere from a congenic transgenic GFP embryo resulted in a visible somatic GFP chimera founder in G0 generation. Mating of the chimeric founder with wildtype non-GFP animals over multiple mating cycles resulted in GFP offspring exclusively in the F₁ generation. This was consistent whether the founder was a male or a female, confirming germline transmission in both male and female background.

We tested and proved the hypothesis that ablation of a PGC specification gene, PRDM14 similarly results in the loss of PGC specification and consequently germ cells in a domestic pig model. Using two separate strategies 1) direct injection of CRISPR ribonucleoproteins (RNP; Cas9 protein complexed with sgRNA) targeting exon 4 of PRDM14 alongside a HDR oligo carrying a “TAG” stop codon into the embryos and transferring the injected embryos into estrus synchronized recipient gilts; and 2) nucleofection of fetal fibroblasts cells with the RNPs and targeting oligos with the stop codon, and culturing the resultant cells at a clonal density to obtain PRDM14 knockout pig fetuses for genotyping and phenotyping analysis.

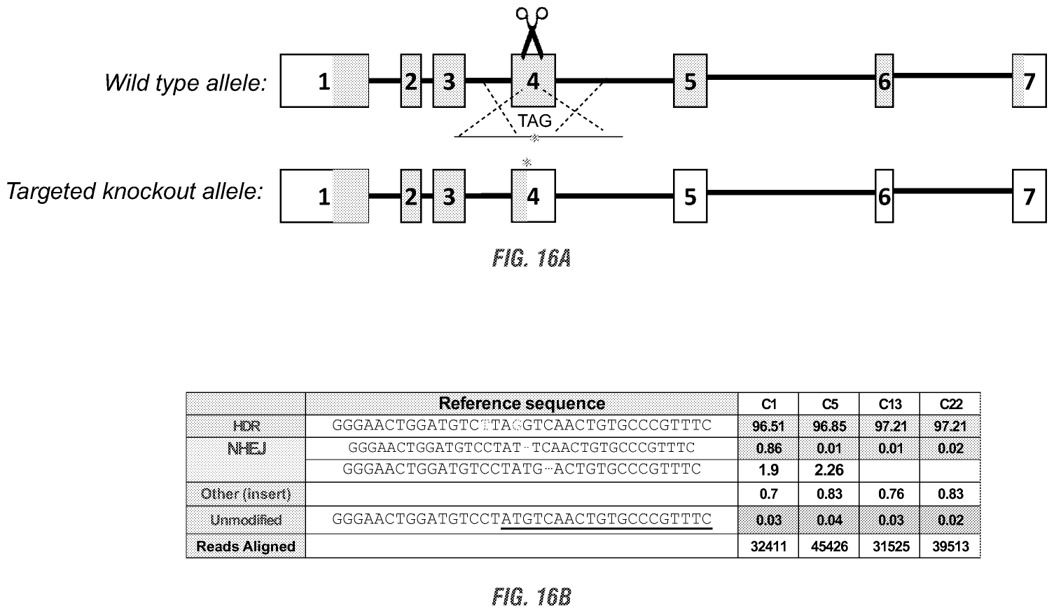

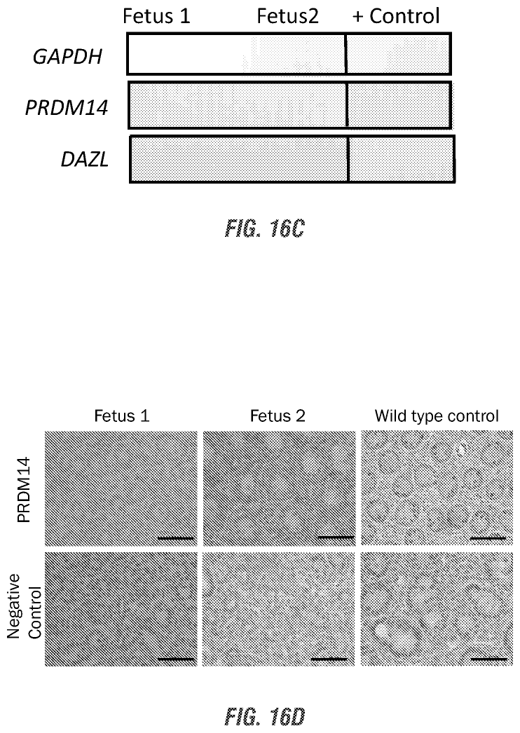

Targeting strategy and results from cellular targeting is shown in FIG. 16A. Briefly, fetal fibroblast cells from a crossbred (landrace X large white) pig were nucleofected using Amaxa Nucleofector 4D system, alongside RNPs targeting exon 4 of PRDM14. Exon 4 was chosen because it is the common exon in all the isoforms and is before the functionally important SET and zinc-finger DNA binding domains of PRDM14 protein. Besides the RNPs, a targeting oligo for insertion of a “TAG” stop codon in frame with the coding sequence was utilized. Following nucleofection with the RNAPs and the targeting oligo, the cells were cultured for an additional two days, and plated at a low density onto a 10 cm dish. Medium in the culture dish was replaced periodically, and the cells were allowed to grow and form colonies in the dish. Using cloning cylinders, clonal lines were obtained and propagated in a 12-well dish, and passaged further into a 6 well dish. At which point, genomic DNA from leftover cells following passaging was harvested, DNA isolated and screened using a high throughput Illumina iSeq platform (targeted amplicon sequencing). Sequencing results from four representative colonies revealed >96-97% reads harboring precise targeted knockin of “TAG” translational stop codon in-frame with the coding sequence, and resulting in the knockout of the gene (FIG. 16C). Two cell colonies (colony #C13 and C22) were used as nuclear donors for somatic cell nuclear transfer (or cloning) to generate PRDM14 knockout embryos and transferred into oviducts of estrus synchronized recipient animals. Following confirmation of successful pregnancy at day 35 of pregnancy when the PGC will have migrated to the gonad and start developing into germ cells, the recipient animals were humanely euthanized, fetuses recovered to collect the gonads. One of the two gonads was used for RNA isolation and screening for loss of expression of germ cell markers (FIG. 16C) and another gonad fixed in 4% paraformaldehyde (PFA) and used for immunohistochemistry (FIG. 16D). Results from these experiments confirmed loss of PGC and consequently germ cells in the PRDM14 null pig fetuses.

### Example 4: Aggregation of PRDM14 Null Embryos with GFP Positive Blastomeres and Transfer into Recipient Animals to Generate Surrogate Sires/Dams

### Somatic Cell Nuclear Transfer of Clonal Lines

In vitro, matured oocytes will be enucleated by aspirating the polar body and MII chromosomes with an enucleation pipette (Humagen, Charlottesville, VA, USA) in 0.1% DPBS supplemented with 5 μg/mL of cytochalasin B. Fetal fibroblasts (FF) from validated PRDM14^{-/-} and a validated donor GFP knockin reporter cells will be synchronized to the G1/G0-phase by serum deprivation (DMEM with 0.2% FCS) for 96 hr. After enucleation, donor cells will be placed into the perivitelline space of an enucleated oocyte. Fusion of cell-oocyte couplets will be performed by applying two direct current (DC) pulses (1-sec interval) of 2.1 kV/cm for 30 s using a ECM 2001 Electroporation System (BTX). After fusion, the reconstituted oocytes will be activated by a DC pulse of 1.2 kV/cm for 60 s, followed by post-activation in 2 mM 6-dimethylaminopurine for 3 hr. After overnight culture in PZM3 with a histone deacetylase inhibitor Scriptaid (0.5 μM), the cloned embryos will be cultured in PZM3 for another two days until the embryos reach 4-8 cell stage.

### Aggregation of Embryos for Generating Chimeric Surrogate Sires and Dams

Donor GFP and recipient PRDM14^{-/-} SCNT embryos at 4-8 cell stage will be used for embryo aggregation. The donating GFP embryo will be denuded by exposure to acid tyrode solution (Sigma Aldrich) for approximately one minute or until the zona pellucida fell off. Individual blastomeres will be disaggregated via vigorous pipetting. Disaggregated donor blastomeres will be treated for 1 hr in phytohemagglutinin (PHA) treatment and injected into 4-cell recipient embryos. Injected embryos will then be washed and incubated in 50 μl drops of PZM medium under mineral oil (Sigma-Aldrich) at 38.5° C. in a trigas incubator with 5% O₂, 5% CO₂, and 90% N₂ air until they reach early blastocyst stage on day 5. Healthy and fully aggregated embryos will then be sorted for transfer into surrogates. Non-aggregated PRDM14 null embryos will be used as controls.

### Embryo Transfers

The recipients for embryo transfers will be synchronized by oral administration of progesterone analog REGU-MATE® (Merck) for 14-16 days. Animals at days 5-6 after natural heat will be used for aggregated blastocyst transfer (into uterus) for generating chimeras. Surgical procedure will be performed under a 5% isofluorane general anesthesia following induction with TKX (Telazol 100 mg/kg, ketamine 50 mg/kg, and xylazine 50 mg/kg body weight) administered intramuscularly. Pregnancies will be confirmed by ultrasound on day 27 following transfer. Fetuses will be recovered on day 35 of pregnancies for harvesting gonads and confirming chimerism and germline contribution. Additionally, pregnancies will be allowed to go term and the piglets will be recovered following natural delivery.

### Fetus Recovery and Screening for Putative Loss of Germ Cells

On day 35 (30 days after embryo transfer), the embryo transfer recipient animals will be humanely euthanized, and fetuses will be recovered for screening chimerism and germline contribution from injected donor GFP embryonic cells. Fetuses will be assessed macroscopically for viability and GFP expression. One of the two gonads will be fixed for immunohistochemical analysis, whereas the second gonad will be utilized for RNA extraction. Fetal tissues will be harvested for DNA extraction. For isolation of genomic DNA (gDNA) from cells and tissues, the QIAamp mini DNA Kit (Qiagen) will be used according to the manufacturers' instructions. Total RNA will be isolated using Trizol plus RNeasy mini kit (Qiagen) and mRNA from individual blastocysts will be extracted using the DYNABEADS™ mRNA Direct Kit (Dynal Asa). Synthesis of cDNA will be performed using a High Capacity cDNA Reverse transcription kit (Applied Biosystems).

The gDNA samples will be subjected to PCR for chimera detection with genotyping primers, and qPCR performed for the detection of GFP allele and chimerism rate. Prior to use in the qPCR analysis, the dynamic range of qPCR primers will be validated (amplification efficiency >90%). The GFP labeled pXEN cell line (Xnt GFP #3-2) will be used as a positive control (GFP+, 100%) and fetal fibroblasts from wild type fetuses will be used as a negative (GFP−, 0%) control for investigating % chimerism. Relative expression will be calculated using the comparative 2^{-ΔΔCt} method. qPCR will be performed in triplicate. Cycling conditions for both GFP and reference (ACTB and YWHAZ gene) products will be 10 min at 95° C., followed by 40 cycles of 95° C. for 15 sec, and 60° C. for 1 min.

RT-PCR using primers against a panel of germ cell markers including but not limited to PRDM1, SALL4, DPPA3, DDX4, KITLG, DAZL, DND1, PRMT5, NANOG, or AID will be performed. Likewise, immunohistochemistry will be performed with antibodies validated towards pig antigens.

### Breeding and Analyzing the Fecundity and Fertility of Surrogate Sires

A few of the embryo transfers from aggregated embryos will be allowed to go to term and farrow naturally. The offspring will be monitored for body condition and reproductive development as below:

Body condition. The animals will be weighed on a bi-weekly basis to screen for body condition and fitness.

Testicular ultrasound. Testes of boars at the immature and adult stages of development will be imaged using an Exago ultrasound machine and static images will be captured to measure the diameter of testes.

Testicular biopsy and cross-sectional analysis. To assess whether seminiferous tubules are intact and the germline is present in surrogate sires, biopsies of parenchyma will be collected for cross-sectioning. Briefly, boars will be placed under general anesthesia, a small incision will be made in the scrotum and an 18 gauge biopsy punch will be inserted into the testicular parenchyma and ˜100 mg of tissue removed. The tissue will then be fixed for 2-3 hours in Bouin's solution followed by washing in 70% ethanol and processing for paraffin embedding. Cross-sections of 5 μm thickness will be adhered to glass slides, deparaffinized, and then stained with hematoxylin and eosin. Histological analysis will be performed to measure the circumference of the seminiferous tubules. Approximately 100 fields will be measured for each sample using Nikon software. Immunohistochemical analysis against known germ cell markers will be performed for monitoring reproductive fitness and germ cell development.

Blood sampling and steroid hormone measurements. To evaluate functionality of the hypothalamic-pituitary gonadal (HPG) axis in surrogate sire/dam pigs, serum testosterone and estrogen concentrations will be measured using LC-MS. Briefly, blood samples will be collected every 15 minutes for 1 hour because testosterone is secreted in a pulsatile manner. Samples will then be centrifuged to separate serum and plasma and the serum stored at −20° C. before shipment to the Endocrine Technologies Support Core (ETSC) at the Oregon National Primate Research Center (ONPRC) for measurement of testosterone by LC-MS analysis.

Semen collection and analysis. Assessment of sperm production by surrogate sires will conducted by collecting semen samples. Briefly, boars will be trained at a young pre-pubertal age on a dummy apparatus (MOFA) for manual semen collection. Samples will be diluted in commercial extender solution (MOFA) and analyzed by light and fluorescent microscopy. Semen from the founders will be FACS sorted for expression of GFP. We expect all the spermatozoal to be GFP positive and therefore of donor origin.

Surrogate gilt estrus detection and insemination. The chimeric founder females will be heat checked daily and estrus activity noted from five-six months of age for regular 21-day cycles. Estrus in the gilts will be detected by noting visible signs of estrus behavior including increased physical activity, phonation, pointed ears, and lordosis (arching of the back in response to physical pressure) in the presence of teaser boar. The gilts will be bred to with PIC semen at 10 months and pregnancies confirmed by ultrasound 28 days later. Fetuses will be harvested to screen for expected GFP expression. This will confirm successful germline transmission.

### Example 5: Generation of Surrogate Sires and Dams by Ablation of Endogenous Germline in Cattle

### Ablation of PRDM14 in Cattle

CRISPR ribonucleoproteins (RNP; Cas9 protein complexed with sgRNA) targeting PRDM14 will be direct injected into embryos, and the injected embryos will be transferred into estrus synchronized recipient heifers. Following confirmation of successful pregnancy, the recipient heifers will be humanely euthanized, and fetuses will be recovered to collect the gonads. One of the two gonads will be used for RNA isolation and screening for loss of expression of germ cell markers and another gonad will be used for immunohistochemistry. Results from these experiments will confirm loss of PGC and consequently germ cells in the PRDM14 null cow fetuses.

### Aggregation of Embryos for Generating Chimeric Surrogate Sires and Dams

Cow embryos will be injected with CRISPR/Cas ribonucleoproteins targeting PRDM14 into the cytoplasm of the embryo to cause knockout of PRDM14. At the 4-8 cell stage, the putative Prdm14^{-/-} embryos will be aggregated with donor-derived embryonic cells or pluripotent cells and transferred into estrus synchronized surrogate recipient heifers.

A few of the embryo transfers from aggregated embryos will be allowed to go to term and give birth naturally. The offspring will be monitored for body condition and reproductive development by testicular ultrasound, testicular biopsy and cross-sectional analysis, blood sampling and steroid hormone measurements, semen collection and analysis, and surrogate heifer estrus detection and insemination. The resulting offspring will be chimeric and will contain both donor and recipient somatic cells, but the germline will be contributed exclusively by the donor cells.

What is claimed is:

1. A method for producing a non-human chimeric embryo with donor-derived germ cells, the method comprising:
providing a host embryo comprising an inactivated primordial germ cell (PGC) specification gene; and
complementing the host embryo with donor cells to yield the chimeric embryo, wherein the germ cells of the chimeric embryo are exclusively derived from the donor.

2. The method of claim 1, wherein the inactivated PGC specification gene is PRDM14.

3. The method of claim 1, wherein the inactivated PGC specification gene is PRDM1, SALL4, IFITM1, DPPA3, DDX4, KITLG, DAZL, DND1, PRMT5, NANOG, AICDA, or TIAL1.

4. The method of claim 1, wherein the host embryo is complemented at the blastocyst stage.

5. The method of claim 1, wherein the host embryo is complemented at the 4-cell stage, 6-cell stage, or 8-cell stage.

6. The method of claim 1, wherein the donor cells comprise one or more pluripotent cells.

7. The method of claim 6, wherein the one or more pluripotent cells comprise embryonic stem cells or induced pluripotent stem cells.

8. The method of claim 6, wherein the one or more pluripotent cells comprise a blastomere of a 4-cell stage donor embryo.

9. The method of claim 1, wherein the animal is a mouse.

10. The method of claim 1, wherein the animal is a pig.

11. The method of claim 1, wherein the animal is cattle.

12. The method of claim 1, wherein the inactivation of the PGC specification gene is accomplished by gene editing.

13. The method of claim 12, wherein the gene editing comprises use of a TALEN, a zinc finger nuclease, or RNA-guided CRISPR-Cas.

14. The method of claim 1, wherein the inactivation of the PGC specification gene is accomplished by injecting a zygote with a Cas protein and a guide RNA that targets the PGC specification gene.

15. The method of claim 1, wherein the donor cells are from an elite animal.

16. The method of claim 1, wherein the donor cells are from an animal with poor breeding performance.

17. A non-human chimeric embryo produced by the method of any one of claims 1-16.

18. The method of any one of claims 1-16, further comprising:
transferring the chimeric embryo into a recipient female animal; and
allowing the transferred chimeric embryo to develop to term as a chimeric animal.

19. The method of claim 18, further comprising:
collecting semen from the chimeric animal.

20. The method of claim 18, further comprising:
breeding the chimeric animal with a second animal to produce one or more progeny animals.

21. The method of claim 20, wherein the breeding comprises natural mating, artificial insemination, or in vitro fertilization.

22. A method for producing a non-human chimeric animal with donor-derived germ cells by blastocyst complementation, the method comprising:
injecting a zygote with a Cas protein and a guide RNA that targets the PRDM14 gene or the PRDM1 gene and allowing the zygote to develop into a blastocyst;
complementing the blastocyst with embryonic stem cells from a donor to yield a chimeric blastocyst, and
transferring the chimeric blastocyst to the uterus of a female recipient animal and allowing a chimeric animal to develop, wherein the chimeric animal comprises germ cells exclusively derived from the donor.

23. A non-human chimeric animal produced by the method of claim 22.

24. A method for producing a non-human chimeric animal with donor-derived germ cells by embryo-embryo aggregation, the method comprising:
injecting a zygote with a Cas protein and a guide RNA that targets the PRDM14 gene or the PRDM1 gene and allowing the zygote to develop into a 4-cell to 8-cell stage embryo;
complementing the embryo with a blastomere from a donor 4-cell stage embryo to yield a chimeric embryo; and
transferring the chimeric embryo to the oviduct of a female animal and allowing a chimeric animal to develop, wherein the chimeric animal comprises germ cells exclusively derived from the donor.

25. A non-human chimeric animal produced by the method of claim 24.

26. A non-human chimeric embryo comprising host cells and donor cells, wherein the host cells comprise an inactivated primordial germ cell (PGC) specification gene, and wherein the donor cells exclusively contribute to the germ cells of the chimeric embryo.

27. The chimeric embryo of claim 26, wherein the inactivated PGC specification gene is PRDM14.

28. The chimeric embryo of claim 26, wherein the inactivated PGC specification gene is PRDM1, SALL4, IFITM1, DPPA3, DDX4, KITLG, DAZL, DND1, PRMT5, NANOG, AICDA, or TIAL1.

29. The chimeric embryo of claim 26, wherein the animal is a mouse.

30. The chimeric embryo of claim 26, wherein the animal is a pig.

31. The chimeric embryo of claim 26, wherein the animal is cattle.

32. A chimeric animal developed from the chimeric embryo of any one of claims 26-31.
